(19) United States
(12) Patent Application Publication
(10) Pub. No.: US 2013/0217037 A1
(43) Pub. Date: Aug. 22, 2013

(54) FUNCTIONALIZATION OF GOLD NANOPARTICLES WITH ORIENTED PROTEINS, APPLICATION TO THE HIGH-DENSITY LABELING OF CELL MEMBRANES

(71) Applicants: Alain Brisson, Arcachon (FR); Stephane Mornet, Bordeaux (FR)

(72) Inventors: Alain Brisson, Arcachon (FR); Stephane Mornet, Bordeaux (FR)

(21) Appl. No.: 13/763,943

(22) Filed: Feb. 11, 2013

Related U.S. Application Data
(63) Continuation of application No. 12/298,510, filed on Oct. 24, 2008.
(60) Provisional application No. 60/794,509, filed on Apr. 25, 2006.

(51) Int. Cl. G01N 33/58; G01N 33/92

(52) U.S. Cl. CPC G01N 33/587; G01N 33/92
USPC 435/7.23; 435/7.32

(57) ABSTRACT

The present invention relates to nanoparticles the surface of which is modified by deposition of proteins. The invention further relates to a method for producing said nanoparticles and to their use in biological research and in the biomedical field (for example labelling and diagnosis).

### FIELD OF THE INVENTION

The present invention relates to nanoparticles the surface of which is modified by deposition of proteins. The invention further relates to a method for producing said nanoparticles and to their use in biological research and in the biomedical field (for example labelling and diagnosis).

### BACKGROUND OF THE INVENTION

Colloidal gold particles of small size, from 1 to 20 nm in diameter, have been used for several decades as specific labels in cell ultrastructure research by Transmission Electron Microscopy (TEM) (1-2). Indeed gold nanoparticles functionalized with antibodies, or other types of biological molecules, allow characterizing the number and the localization of cellular antigens, or other types of complementary elements, in thin sections or in homogenized suspensions of cells or tissues, according to classical techniques of TEM. Classical methods for immobilizing antibodies—or other types of proteins or molecules—on gold particles are based on their direct, non specific and non covalent adsorption, with the exception of the covalent coupling of molecules presenting an accessible sulfhydryl (thio, SH) group. The direct and non-covalent adsorption of molecules on gold particles is often called physical adsorption or physisorption. It is indeed well-known that proteins adsorb in a non covalent manner to the vast majority of inorganic or organic surfaces, the most classical example being the immobilization of proteins on plastic supports in Enzyme-Linked Immuno-Sorbent Assay (ELISA) used in many diagnosis tests. The physical adsorption of molecules, e.g. proteins, on a solid support results from the formation of weak bonds between the molecule and the substrate, these bonds corresponding to electrostatic, van der Waals, hydrogen or hydrophobic interactions. The direct adsorption of macromolecules on solid supports has the main advantage of being simple and applicable to almost any type of macromolecules. However, physical adsorption presents severe limitations as the interactions involved may lead to molecular rearrangements, which may result in a partial denaturation in the case of proteins and in a loss of their biological properties. The direct adsorption approach presents several other severe limitations, principally the absence of binding specificity, as in theory any molecule can bind, and the lack of control of the orientation of molecules bound to the surface. In the case of gold nanoparticles, or other colloidal particles, the question of the colloidal stability of the particles constitutes an additional issue. Indeed, bare nanoparticles are in general unstable in physiological solutions. The physical adsorption of proteins is in general not sufficient to stabilize nanoparticles. This is why stabilizing agents, like bovine serum albumin (BSA) or surfactants, are present in most commercial suspensions of gold-protein conjugates. The presence of these additives is however problematic as they may interfere with the molecular processes investigated or perturb the integrity of the studied cellular structures, as it is well proven for surfactants.

In addition, the physical adsorption of proteins on gold nanoparticles is irreproducible, inefficient and tedious, as it must be optimized for every new protein to be coupled. Therefore this approach is costly in time and costly in products, as it requires large amounts of proteins, in general of high value. Consequently, the commercially available protein-functionalized gold particles contain only low amounts and low concentrations of products.

The multiple limitations associated with the physical adsorption approach explain the development of alternative approaches for coupling biological molecules to solid supports in a controlled manner.

Diverse strategies for coupling covalently peptides or proteins to the surface of metal nanoparticles (gold, silver, platinum, palladium . . . ) have been reported (3-14). Many of these studies make use of spacers to separate the metal particles from the biological moiety, to avoid their possible denaturation. Most often, the spacers are covalently linked at one end to the gold nanoparticle via thiol chemistry, while they are linked at the other end, to amine groups exposed at the protein surface, e.g. via use of N-hydroxysuccinimide (NHS)-containing ligands. As every protein presents several accessible amine groups, this coupling approach results in a random and multiple orientation of proteins at the surface of gold particles and is non specific. In addition, amine groups often participate in active sites or ligand-binding sites, and their modification may lead to loss of recognition properties. Several studies describe the coupling of spacers to sulfhydryl- or thiol-(SH) groups exposed at the protein surface, as for example in the case of Fab′-SH antibody fragments (5, 14). This approach has limited application, because only few proteins present accessible SH-groups, even after disulfide reduction. In addition, the production of Fab′-SH proteins is not straightforward, requiring experts in the art, and has low yield. Furthermore, although it is possible to transform amine groups into sulfhydryl groups, as for example with use of the Traut's reagent, the use of amine groups results in random orientation of coupled proteins, as discussed above. In conclusion, no reliable strategy has yet been proposed for controlling the orientation of proteins linked to gold particles, in such a way that the sites complementary to the target molecule of interest are properly exposed to the aqueous environment.

In this context, there is a need to develop a reliable and general method for coupling proteins to nanoparticles in a specific manner, with controlled orientation and density, and to produce suspensions of protein-functionalized particles of high stability and high concentration.

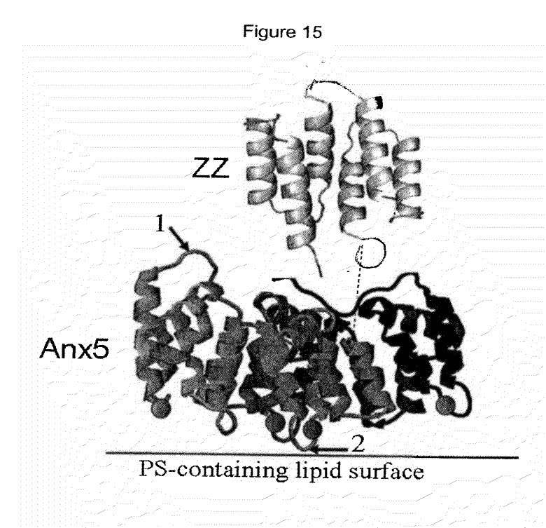

Annexin-A5 (Anx5) is a soluble protein, of about 35 kDa, which presents the property to bind to negatively charged phospholipids, like phosphatidyl-serine (PS) in the presence of calcium ions (15-17). Anx5 is widely used as a marker of the physiological processes of platelet activation and apoptosis, or programmed cell death (18-19). These processes are characterized by a membrane reorganization resulting in the exposure of PS molecules on the outer layer of the plasma membrane. Assays have been developed for characterizing platelet activation or apoptosis, which are based on labelling PS-containing membranes with fluorescently-labelled Anx5 molecules (20, 21). Modified Anx5 proteins, including fusion proteins and mutant Anx5 proteins, have been recently described (22). A preferred example of said Anx5 derivatives is made of mutant Anx5 proteins that present one single sulfhydryl group, like for example the one referred to hereafter as Anx5-[T163C;C314S]; in said specific mutant, the naturally occurring C314 has been replaced by a serine residue, and the residue T163 located in a solvent-exposed loop on the concave face of Anx5 opposed to the membrane-binding face has been mutated to a cysteine (FIG. 15). This strategy of site-directed mutagenesis has for objective to create a reactive group, namely a —SH group from a cysteine, at a selected position within the protein structure, in order to allow subsequent coupling of molecular entities presenting groups able to react with —SH groups. The mutant Anx5 [T163C;C314S] protein presents all known properties of wt Anx5 (22). Another example of said mutant Anx5 protein with one single sulfhydryl group is Anx5 [A260C;C314S], in which the sulfhydryl group is exposed on the membrane-binding face. Another preferred example of said modified Anx5 proteins, is made of Anx5-Z or Anx5-ZZ fusion proteins, by recombinant DNA technology (23, 24), as described in (22) (FIG. 15). The Z domain is a protein domain homologous to the B domain of protein A from Staphylococcus aureus, which is responsible for the affinity of protein A for the Fc fragment of antibodies. Anx5-Z and Anx5-ZZ fusion proteins combine the properties of their two halves, namely the property of their Z, or ZZ, moiety to bind specifically to IgGs, and the properties of their Anx5 moiety to bind to PS-containing membrane surfaces, to form trimers upon binding to PS-containing membrane surfaces, and to form two-dimensional crystals of trimers on PS-containing lipid monolayers and lipid bilayers supported on mica (17).

Gold nanoparticles coupled to Anx5 have already been produced by the physical adsorption approach and are commercialized by Bio-VAR (Armenia). Said nanoparticles have the limitations of physical adsorption reported above: lack of colloidal stability, necessary presence of stabilizing agents, protein denaturation, no control of protein orientation, and low concentrations of functionalized nanoparticles.

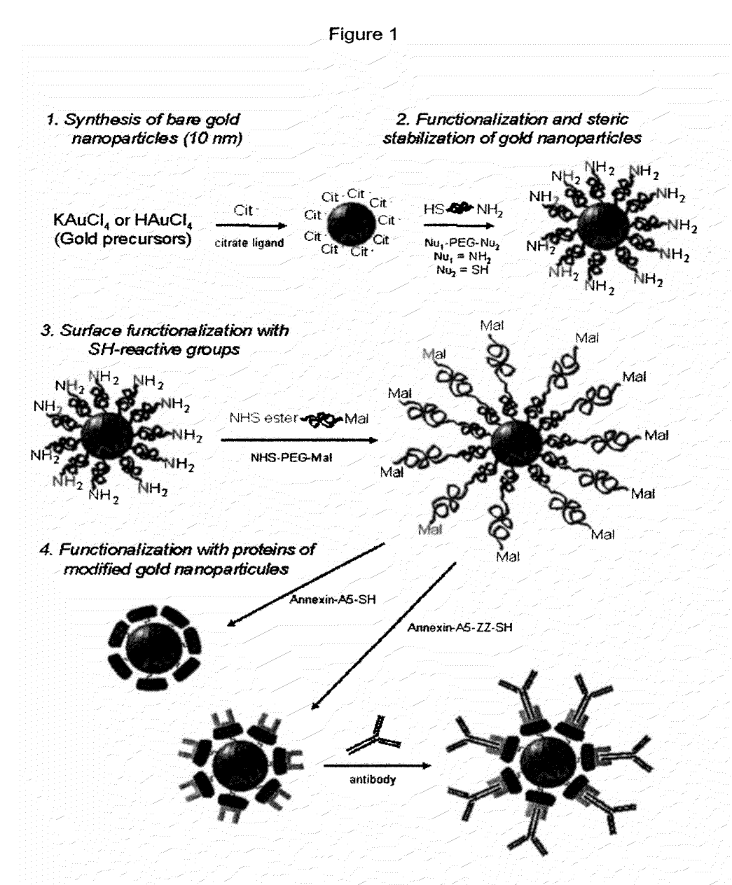

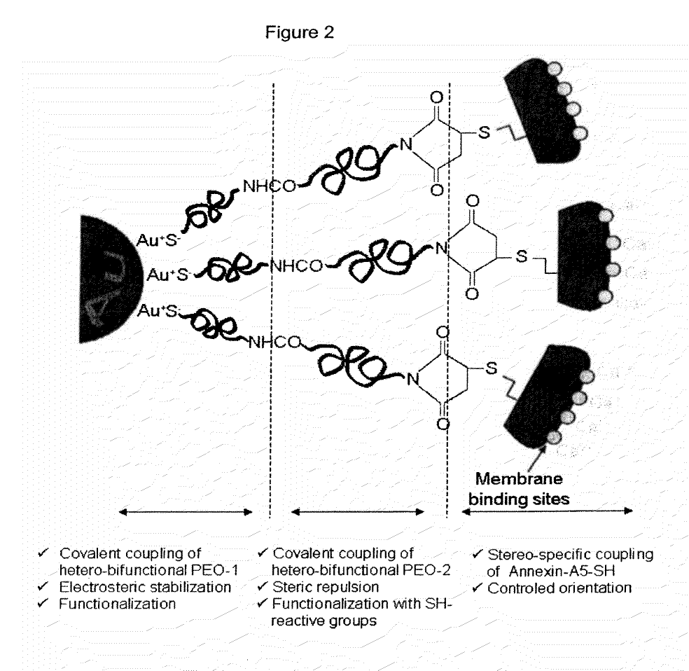

The inventors have now discovered that it was possible to synthesize nanoparticles functionalized with proteins with controlled orientation and density. In the case of Anx5, the proteins are oriented with their convex membrane-binding face exposed to the aqueous solution (as schematized in FIGS. 1 and 2). In the case of Anx5-Z or Anx5-ZZ fusion proteins, the protein is oriented with the Z or ZZ fragments exposed to the aqueous solution (FIG. 1).

### DETAILED DESCRIPTION OF THE INVENTION

The present invention relates to surface functionalized nanoparticles with a size comprised between 1 nm and 1 μm, preferably 1 to 20 nm, more preferably 10 nm, having a surface modified by grafting thereon by covalent linkage a plurality of spacers, a spacer being itself linked to a protein in a stereo-specific manner, ensuring controlled orientation of the particle-bound protein.

In the present invention, the terms linker and spacer are used undifferently.

According to an embodiment of the invention, the spacers are selected from the group comprising homo-bifunctional polyethylene oxides, hetero-bifunctional polyethylene oxides, home- or hetero-bifunctional polyethylene oxide containing linkers, homo- or hetero-polypeptides, or functionalized oligonucleotides.

According to an embodiment of the present invention, linking the spacer to the protein covalently is performed preferably by linking a spacer terminated by a —SH reactive group to a protein presenting one accessible thiol (—SH) group of a cysteine.

According to an embodiment of the present invention, linking the spacer to the protein by affinity is performed preferably by linking a spacer terminated by a Ni-NTA (Nickel II-nitrilotriacetic acid) group to a protein presenting a poly-histidine extension, or by linking a spacer terminated by a biotin group to a streptavidin, itself linked to a protein presenting a biotin group.

According to an embodiment of the invention, said spacer consists of one or several, preferably two, covalently-linked spacers selected from the group comprising homo- or hetero-bifunctional polyethylene oxides. Advantageously, said spacer consists of two covalently linked homo- or hetero-bifunctional polyethylene oxide spacers, the first spacer being covalently linked to the nanoparticle and the second spacer being covalently linked to the first spacer at one end and linked to the protein at the other end.

According to an embodiment of the present invention, the nanoparticles are covalently modified with a plurality of hydrophilic homo- or hetero-bifunctionnal polyethylene oxide spacer, said spacers being themselves covalently linked to an homo- or hetero-bifunctionnal polyethylene oxide spacer, being itself coupled to a protein presenting one accessible thiol group, in a covalently and stereo-specific manner, ensuring specific orientation to the particle-bound protein.

In the present invention, the terms (polyethylene oxide (PEO) and polyethylene glycol (PEG) are used undifferently for designing polyethylene oxide moieties of the spacers.

According to an embodiment of the invention, the nanoparticles are gold nanoparticles; other metallic clusters like silver, platinum, palladium, iron-gold alloy, iron-platinum alloy, and transition metal chalcogenides passivated by zinc sulfide, whatever is their form (spherical, faceted or rod-like).

According to one embodiment of the present invention, the protein presenting one accessible thiol group has particular affinity for anionic phospholipids or for other membrane-associated components.

Said anionic phospholipids or other membrane-associated components may be advantageously selected from the group comprising phosphatidyl-serine, phospatidic acid, phospatidyl-glycerol, any other negatively charged phospholipid, and any negatively charged lipid at neutral pH.

According to the invention, the protein having particular affinity for anionic phospholipids or other membrane-associated components and presenting one accessible thiol group is selected from the group comprising annexins, coagulation factors, phospholipid-binding antibodies, phospholipases, lactadherin, proteins containing one or several membrane-binding C2 domains, or any protein binding to a lipid surface containing molecules from the group comprising phosphatidyl-serine, phosphatidic acid, phosphatidyl-glycerol, any other negatively charged phospholipid, and any negatively charged lipid at neutral pH.

According to the instant invention, annexin is selected from the group consisting of Annexin-A1, Annexin-A2, Annexin-A3, Annexin-A4, Annexin-A5, Annexin-A6, Annexin-A7, Annexin-A8, Annexin-A9, Annexin-A12, Annexin-A, Annexin-B, Annexin-C and Annexin-D, as well as anyone of their annexin derivatives.

In the present invention, a protein derivative means a natural protein which has been modified but which is still functionally active despite said modifications, which means that this protein derivative still has the properties of the natural protein from which it is derived. For example, when the protein derivative is an annexin derivative, this annexin derivative still has particular affinity in presence of calcium ions for anionic phospholipids or for other membrane-associated components. Modifications of the natural protein to obtain the protein derivative may consist for example in mutation(s) and/or fusion with another polypeptide or protein, as well as addition of a poly-histidine extension or a biotin group.

The functionally active derivatives of annexin-A5 exhibit the characteristic properties of annexin-A5, principally they have particular affinity in presence of calcium ions for anionic phospholipids or for other membrane-associated components, they form trimers upon binding to a PS-containing membrane surface and they form two-dimensional crystals of trimers on lipid monolayers and on lipid bilayers supported on mica (17).

Also in the present invention, a modified stereo-specifically protein derivative is a protein derivative presenting an accessible thiol (—SH) group or an accessible poly-histidine extension or an accessible biotin group at a site which is preferably opposed to the binding or active site of the protein and which is accessible for linkage to the nanoparticles via the spacers. Said groups are inserted by any technique well-known from the one skilled in the art. For example the —SH group may be inserted by replacing one amino-acid of the protein by a cysteine.

In the present invention, the terms stereo-selectively and stereo-specifically are used undifferently.

In a specific embodiment, Annexin-A5 is from a species selected from the group consisting of Rattus, Homo sapiens, Mus, Gallus and Bos, as well as any one of their annexin derivatives.

In another specific embodiment, the annexin derivative is a mutant annexin containing one single cysteine with accessible thiol group and/or an annexin derived fusion protein which binds to the Fc fragment of antibodies. More preferably, the annexin derivative is a double mutant Annexin-A5 from Rattus norvegicus containing the mutation C314S and a mutation selected from the group consisting of T163C, A164C, I165C, A2C and any other mutation resulting in one accessible thiol group.

In another advantageous embodiment of the instant invention, the double mutant Annexin-A5 is the naturally occurring Annexin-A5 from Rattus norvegicus having the mutations C314S and T163C.

According to a particularly advantageous embodiment of the invention, the nanoparticles are gold nanoparticles, of size ranging between 1 nm and 50 nm (preferably near to 10 nm), functionalized with homo- or hetero-bifunctional poly(ethylene oxide) (PEO) molecules and coupled, covalently and stereo-selectively, to proteins derived from Anx5. The proteins derived from Anx5 used in this invention were the subject of the international application WO2005114192 (22). In particular, the double mutant Anx5 [T163C;C314S] which presents a unique SH group site-specifically inserted presents all the known properties of native Anx5 of binding to lipidic surfaces and consequently the double mutant Anx5 [T163C, C314S] is called Anx5-SH hereunder. In particular also, the Anx5-ZZ fusion proteins containing either one of the double mutants Anx5 [T163C;C314S] or Anx5 [A260C;C314S] presents all known properties of Anx5-ZZ fusion proteins and do not present noticeable differences between each other. They will be called Anx5-ZZ-SH proteins hereunder where Z is a protein domain homologous to the B-domain of protein A from Staphylococcus aureus.

In another embodiment, the annexin derivative is the annexin derived fusion protein selected from the group comprising Annexin-Z fusion protein and Annexin-ZZ fusion protein, where Z is a fragment of protein A from Staphylococcus aureus. Advantageously, the Annexin-Z fusion protein and the Annexin-ZZ fusion protein contain Annexin-A5 double mutant from Rattus norvegicus having a double mutation selected from the group comprising [T163C;C314S], [A260C;C314S], [W185C;C314S], [G259C;C314S], [G261C;C314S], [G28C;C314S], [L429C;C314S], [G30C;C314S], [G100C;C314S], [A101C;C314S], [G102C;C314S], [G186C;C314S] and [T187C;C314S].

A further embodiment of the invention is Surface functionalized nanoparticles wherein the first homo- or hetero-bifunctional polyethylene oxide (PEO or PEG) spacer has the formula (1)

Nu₁-PEG-Nu₂ (1)

wherein Nu₂ represents a nucleophilic group able to be covalently linked to the surface of the nanoparticle and selected from the group comprising —SH group and other gold reactive groups, and Nu₁ represents a nucleophilic group selected from the group comprising —SH, —NH₂ and —OH groups.

In another embodiment, the second homo- or hetero-bifunctional polyethylene oxide spacer presents at one end a group able to react with —SH, —NH₂ and —OH group, and at the other end a thiol reactive group able to react with the thiol group of a cysteine of the protein. Preferably, the second hetero-bifunctional polyethylene oxide spacer is selected from the group comprising NHS-PEG-Mal and vinylsulfones (VS) derivated PEOs such as NHS-PEG-VS.

In a further embodiment, the hetero-bifunctional polyethylene oxide spacer Nu₁-PEG-Nu₂ has a molar mass higher than 300 g/mol and the second hetero-bifunctional spacer is selected from the group comprising N-Succinimidyl 3-[2-pyridyldithio]-propionamido (SPDP), Succinimidyl 6-(3-[2-pyridyldithio]-propionamido)hexanoate (LC-SPDP), 4-Succinimidyloxycarbonyl-methyl-a-[2-pyridyldithio]toluene (SMPT), 4-Sulfosuccinimidyl-6-methyl-a-(2-pyridyldithio)toluamido]hexanoate) (Sulfo-LC-SMPT), Succinimidyl 4-[N-maleimidomethyl]cyclohexane-1-carboxylate (SMCC), Succinimidyl 4-[N-maleimidomethyl]cyclohexane-1-carboxy-[6-amidocaproate] (Sulfo-SMCC), m-maleimidobenzoyl-N-hydroxysuccinimide ester (MBS), m-maleimidobenzoyl-N-hydroxysulfosuccinimide ester (Sulfo-MBS), succinimidyl 4-[p-maleimidophenyl]butyrate (SMPB), Sulfosuccinimidyl 4-[p-maleimidophenyl]butyrate (Sulfo-SMPB), N-[g-maleimidobutyryloxy]succinimide ester (GMBS), N-[g-maleimidobutyryloxy]sulfosuccinimide ester (Sulfo-GMBS), N-e-maleimidocaproyloxy]succinimide ester (EMCS), N-e-maleimidocaproyloxy]sulfosuccinimide ester (Sulfo-EMCS), N-Succinimidyl S-acetyl(thiotetraethylene glycol), (1,4-bis-maleimidobutane (BMB), 1,4 bis-maleimidyl-2,3-dihydroxybutane (BMDB), bis-maleimidohexane (BMH), dimethyl pimelimidate.2 HCl (DMP), bis[sulfosuccinimidyl] suberate (BS³).

In a further embodiment, the nanoparticles are gold nanoparticles which are functionalized by a first polyethylene oxide spacer containing a terminal thiol group (Nu₂=SH) and the second spacer is selected from the group of homo-bifunctional polyethylene oxide comprising bis-maleimides (Mal-PEG-Mal), bis-orthopyridyldisulfides (OPSS-PEG-OPSS) and bis-vinylsulfones (VS-PEG-VS).

In another further embodiment, the nanoparticles are gold nanoparticles, which are functionalized by a first polyethylene oxide spacer having a molar mass higher than 300 g/mol and containing a terminal thiol group (Nu₂=SH) and the second polyethylene oxide spacer is selected from the group of homo-bifunctional bis-maleimide coupling agents comprising α,ω-bis-maleimido(di-, tri- or tetra-) ethyleneglycol).

The invention also encompasses a method of functionalization of gold particles with proteins with controlled orientation and controlled density. One object of the invention is thus a method for obtaining surface functionalized nanoparticles according to the present invention, ensuring controlled orientation and controlled density of the proteins.

In one embodiment the fixation of the spacers is entirely covalent and therefore the resulting protein-gold nanoparticles assemblies are chemically stable. The used strategy allows preserving the structural and functional integrity of the protein, and provides colloidal stability to the protein-functionalized gold nanoparticles. The method allows producing suspensions of gold nanoparticles functionalized by covalent and stereo-specific coupling of proteins, in large quantities and high concentrations, which are stable in physiological medium without the addition of stabilizing agents. The high concentrations (from 0.1 to 5 μM for 10 nm-diameter particles) and the large quantities (50 nmoles) of these suspensions allow to apply the functionalized nanoparticles to the study of the distribution, the localization, or the quantification of the target elements present either in solutions or on sections of biological material in saturating conditions of markers.

In a specific embodiment according to the instant invention the method for obtaining nanoparticles comprises the following steps:
a) optionally, preparation of the nanoparticles,
b) functionalization of the nanoparticles by fixing by a covalent linkage a plurality of spacers,
c) optionally, purification of the functionalized nanoparticles obtained in step b), in order to eliminate the spacers in excess,
d) coupling on said spacers, by covalent or by affinity linkage, a stereo-specific protein derivative having particular affinity for anionic phospholipids or other membrane-associated components, and
e) optionally, purification of the functionalized nanoparticles obtained in step d).

All the steps a) to f) are advantageously realized in an aqueous medium.

The elimination of the polymer (spacer) in excess can be accomplished by any methods known by one skill in the art like for example ultrafiltration, ultracentrifugation or purification on exclusion column of Sephadex® type. Several cycles of washing with ultrapure water have to be carried out so that the maximum residual polymer concentration does not exceed 10⁻⁷ mol/L.

The strategy of synthesis was chosen with the following rationale: 1) to preserve the structural and functional properties of the proteins to be coupled, the chemical reactions must be performed in aqueous medium; 2) to satisfy this constraint, gold nanoparticles were first functionalized with hydrophilic homo- or hetero-bifunctional poly(ethyleneoxide) macromolecules, ensuring the stability of the nanoparticles in saline solutions; 3) to allow the covalent and stereo-specific coupling of SH-exposing proteins, the PEO-functionalized gold nanoparticles must be terminated with SH-reactive groups well known from one skill in the art like for example maleimide (MAL) or vinylsulfone (VS) or dithiopyridine.

The presence of PEO molecules on the surface of gold nanoparticles allows to ensure the colloidal stability of the system in solutions of high salinity, e.g. in physiological medium. Indeed, the hydrophilic chains of PEO molecules ensure steric repulsions maintaining the particles distant from each other. The presence of this macromolecular layer covering the nanoparticle surface has a twofold beneficial effect: it prevents the coalescence or flocculation of the gold nanoparticles (capping agent) and it prevents the non specific adsorption of proteins or other molecules.

The overall scheme of synthesis is presented in FIG. 1.

The first step is the synthesis of bare colloidal particles by reduction of tetrachloroaurate salts in the presence of sodium citrate, according to well-established procedures (25-27).

The second step consists in functionalizing the bare nanoparticles by homo- or hetero-bifunctional PEO macromolecules, of formula (1)

Nu₁-PEG-Nu₂ (1)

wherein Nu₁ and Nu₂ represent nucleophilic function, for example a gold reactive group, preferably a sulfhydryl function, able to carry out a covalent bond of donor-acceptor type with the surface of the gold nanoparticles (27). The formulation can be declined starting from mixtures of hetero-bifunctional PEO (HS-PEO-Nu₂) and PEO terminated by an alcohol group. At this stage, for macromolecules having a sufficient molar mass—or size—, the nanoparticles become stabilized via steric stabilization. For example, for 10 nm-diameter gold nanoparticles, steric stabilization is obtained for molar masses higher than 1000 g/mol.

An alternative method consists in synthesizing the functionalized gold nanoparticles in only one step, by reduction of auric salts with sodium borohydride in the presence of hetero-bifunctional PEO (28, 32). This latter method allows encompassing a wide range of particle sizes going from gold clusters (<1 nm) to nanoparticles with tens of nanometers diameter, depending of the concentration in PEO and auric salts.

Bare nanoparticles can also be functionalized by hetero-bifunctional PEOs in two steps, first by surface modification with ligands containing sulfhydryl and carboxylic functions like tiopronin (5, 6) or the lipoïc acid (7, 8), aminoacids or oligopeptides (9-12) followed by covalent coupling with carboxylic acid groups via the EDC/NHS (1-ethyl-3-[3-dimethylaminopropyl]carbodiimide hydrochloride/N-hydroxysuccinimide) chemistry through the primary amine function of hetero- or homo-bifunctional PEO.

The third step of the synthesis consists in coupling a homo-bifunctional or hetero-bifunctional agent able to react with the terminal nucleophile (Nu₂) on the surface of the functionalized nanoparticles. The classical hetero-bifunctional coupling agents such as N-Succinimidyl 3-[2-pyridyldithio]-propionamido (SPDP), Succinimidyl 6-(3-[2-pyridyldithio]-propionamido) hexanoate (LC-SPDP), 4-Succinimidyloxycarbonyl-methyl-a-[2-pyridyldithio]toluene (SMPT), 4-Sulfosuccinimidyl-6-methyl-a-(2-pyridyldithio)toluamido]hexanoate) (Sulfo-LC-SMPT), Succinimidyl 4-[N-maleimidomethyl]cyclohexane-1-carboxylate (SMCC), Succinimidyl 4-[N-maleimidomethyl]cyclohexane-1-carboxy-[6-amidocaproate] (Sulfo-SMCC), m-maleimidobenzoyl-N-hydroxysuccinimide ester (MBS), m-maleimidobenzoyl-N-hydroxysulfosuccinimide ester (Sulfo-MBS), succinimidyl 4-[p-maleimidophenyl]butyrate (SMPB), Sulfosuccinimidyl 4-[p-maleimidophenyl]butyrate (Sulfo-SMPB), N-[g-Maleimidobutyryloxy]succinimide ester (GMBS), N-[g-maleimidobutyryloxy]sulfosuccinimide ester (Sulfo-GMBS), N-e-maleimido-caproyloxy]succinimide ester (EMCS), N-e-maleimido-caproyloxy]sulfosuccinimide ester (Sulfo-EMCS), N-Succinimidyl S-acetyl(thiotetraethylene glycol) (PEO₄-SATA, after deprotection with hydroxylamine) etc. (Pierce Biotechnology, the USA) can be used within the framework of the formulations for which Nu₂ is a primary amine or when the particles are partially coupled with PEO like HS-PEO-OH. Coupling agents containing carboxylic functions bound to maleimide groups like the maleimidocaproïc acid can react on these amines via EDC or EDC/NHS chemistry. In this formulation the primary amine can also be transformed into sulfhydryl by the 2-imminothiolane (Traut's reagent), the N-succinimidyl-S-acetylthioacetate (SATA) or the N-Succinimidyl-S-acetylthiopropionate (SATP) (after deprotection with hydroxylamine). Homo-bifunctional coupling agents like (α,ω-bis-maleimido (di-, tri- or tetra-)ethyleneglycol (BM(POE₂), BM(POE₃) or BM(POE₄)) (Pierce Biotechnology, the USA) as well as coupling agents using divinyl sulfones, dipyridyldisulfides, dibromo- or diiodoacetyls, dibromo or diiodoalcanes can also be used. In the case of homo-bifunctional coupling agents as second linker, then steric stabilisation is obtained if the first PEOs linker has a mass higher than 300 g/mol.

In the case of the formulation using 100% of hetero-bifunctional PEOs (Nu₁-PEO-Nu₂) with Nu₁=HS and Nu₂=NH₂, the grafting of a coupling agent on the nanoparticle surface, under saturating conditions, requires the use of hydrophilic coupling agents in order not to disturb the stability of the dispersion. The homo-bifunctional and hetero-bifunctional macromolecules commercialized by Nektar (USA) or Pierce Biotechnology companies satisfy this condition because of the high solubility of the PEO chains in aqueous medium.

Homo-bifunctional PEOs able to react specifically with thiols (after transformation of Nu₂ into thiol) include the bis-maleimides (Mal-PEG-Mal), bis-orthopyridyldisulfides (OPSS-PEG-OPSS), bis-vinylsulfones (VS-PEG-VS) (Nektar) or BM(POE2), BM(POE3) and BM(POE4) (Pierce Biotechnology). When Nu₂ is a primary amine, the use of hetero-bifunctional PEOs is better suited because the possibilities of inter-particle coupling are eliminated. In this case, the NHS-PEG-Mal, VS-PEG-NHS (Nektar) or NHS-PEOₙ-Maleimide (n=2, 4, 8, 12; Pierce Biotechnology) coupling agents are preferred.

The fourth and final step of the synthesis consists in the covalent coupling of proteins presenting an accessible thiol group to functionalized gold nanoparticles. For Anx5, it uses the original properties of chimerical proteins described in the patent application WO2005114192 (22).

The strategy of bio-functionalization of the gold nanoparticles is valid for any protein, peptide or molecule presenting an accessible sulfhydryl group, in particular fusion proteins of Anx5-X type, such as Anx5-Z or Anx5-ZZ fusion proteins, where the Z domain is homologous to the B domain of protein A of Staphylococcus aureus, which is responsible of the affinity of protein A for the Fc fragment of IgG antibodies (23, 24). The essential advantage of these proteins lies in the control of the position of the sulfhydryl function obtained by a mutation of an aminoacid to a cysteine. This allows the covalent and stereo-specific coupling and the controlled orientation of proteins at the surface of functionalized gold nanoparticles.

The number, or density, of proteins per gold nanoparticle can be controlled at the fourth step is adjusting the respective concentrations of gold nanoparticles and of proteins. For example, the number of annexin-A5 molecules coupled stereo-specifically per gold nanoparticle of 10 nm diameter can be varied between 1 and 10, 10 corresponding to the maximal density.

The invention also relates to the functionalized gold nanoparticles obtained by such specific method.

The invention also relates to aqueous dispersion containing nanoparticles as described before.

The instant invention also relates to the use of said nanoparticles in biological research or in the biological field.

The nanoparticles according to the instant invention present a high specificity of binding and a high density. Consequently they may be used for labelling, from basic science to medicine, with particular interest in the fields of haematology, oncology and cardiology.

The nanoparticles according to the invention may also be used for investigating any physiological or pathological processes involving a membrane reorganization with the exposure of PS molecules, such as apoptosis, the process of platelet activation in blood coagulation, (33, 34), or the process of mastocyte degranulation characteristic of asthma (35), and any other process characterized by the emission of PS-containing microparticles.

Consequently the instant invention also relates to a method for detecting cells or cell fragments exhibiting a physiological or pathological state involving membrane reorganization with the exposure of phosphatidyl-serine (PS) molecules, the said method including:

a) coupling of surface functionalized nanoparticles according to the instant invention to the cells or cell fragments;
b) detecting the presence of said functionalized nanoparticles coupled to the cells or cell fragments.

Advantageously, when the protein is annexin, the coupling in step a) is made in the presence of calcium ions.

In a preferred embodiment, the step b) of detecting the presence of functionalized nanoparticles coupled to the cells or cell fragments consists in imaging by electron microscopy the cells or cell fragments which have been coupled to said nanoparticles.

It also relates to a method for labelling cells or cell fragments exhibiting a physiological or pathological state manifesting itself by the presence of a receptor for annexin, especially PS, at their surface, the said method including:

a) coupling of particles according to claims 1 to 21 to the cells or cell fragments in the presence of calcium ions;

b) imaging the cells or cell fragments which have been labelled by said nanoparticles by electron microscopy.

The nanoparticles according to the instant invention present several properties that render them adapted to various detection methods. First, they present a high electron scattering cross section, which is at the origin of their use in Transmission Electron Microscopy (TEM) and Scanning Electron Microscopy (SEM) in immuno-labelling studies. In addition, they present interesting optical and spectroscopic properties, as well as properties of interaction with X-rays or with other types of radiation, which allow various approaches of detection and quantification to be developed, among which optical microscopy, spectrophotometry, surface plasmon resonance (SPR), localized surface plasmon resonance (LSPR), Surface Enhanced Raman Scattering (SERS). The high density of labelling constitutes a direct advantage for LSPR or SERS methods. This allows considering the use of these new tools for in vitro and in vivo labelling at the same time. The use of gold nanoparticles in X-ray tomography imaging (p-scanner X) or the application of the developed strategy for developing other types of functionalized particles acting as contrast agents in Magnetic Resonance Imaging (MRI) or radioactively labelled with F18 or Tc⁹⁹ type for positron emission tomography imaging (PET) open multiple possibilities of functional imaging and is of considerable interest in the biomedical field.

The instant invention also relates to a method for diagnosing a physiological or pathological state in an individual comprising the following steps:

a) contacting a biological sample of said individual with surface functionalized nanoparticles according to claims 1 to 21,

b) detecting whether a complex is formed and

c) correlating the formation of said complex with a physiological or pathological state.

According to the instant invention, the physiological or pathological state may be selected from the group comprising a haematological state, a disease involving apoptosis, like cancer, cardiac or neurodegenerative diseases and asthma as well as any state involving membrane reorganization with the exposure of PS molecules.

The instant invention also relates to a method for diagnosing a physiological or pathological state in an individual by saturating the surface of cells or cell fragments with protein-functionalized gold particles according to said invention and detecting the amount of bound nanoparticles or the amount of unbound nanoparticles.

The instant invention also relates to a method for detecting a target molecule in a biological sample, comprising the steps of:

a) contacting a biological sample with nanoparticles according to the instant invention and functionalized with a fusion complex between a protein and bait molecule which bind to said target molecule,

b) detecting the complexes that are formed between the bait moiety of said fusion complex and the target molecule when said target molecule is present in said sample and

c) correlating the formation of said complex with a physiological or pathological state.

The invention also consists in a method for detecting a target molecule in a biological sample, comprising the steps of:

a) contacting a biological sample with nanoparticles according to the invention which are functionalized with a fusion complex between an Annexin-Z derived fusion protein or an Annexin-ZZ derived fusion protein and an antibody, wherein the Z- or ZZ-domain is linked by affinity to the Fc fragment of the antibody, and wherein said antibody is able to bind with said target molecule,

b) detecting the complexes that are formed between the nanoparticles functionalized with the fusion complex and the target molecule when said target molecule is present in said sample, and

c) correlating the formation of said complex with a physiological or pathological state.

Advantageously, the Annexin-Z fusion protein and the Annexin-ZZ fusion protein contain Annexin-A5 double mutant from Rattus norvegicus having a double mutation selected from the group comprising [T163C;C314S], [A260C;C314S], [W185C;C314S], [G259C;C314S], [G261C;C314S], [G28C;C314S], [L29C;C314S], [G30C;C314S], [G100C;C314S], [A101 C;C314S], [G102C;C314S], [G186C;C314S] and [T187C;C314S].

The antibodies are thus linked to the nanoparticles in a controlled orientation, thanks to the stereo-specific linkage of the annexin derivative.

In addition, the density of the antibodies linked to the nanoparticles can be controlled by adjusting the respective concentrations of nanoparticles and of antibodies.

In another advantageous embodiment, Anx5-Z (or -ZZ) functionalized gold nanoparticles with different sizes are advantageous for multiple detection of several target molecules in the same biological sample.

The instant invention is further illustrated by examples 1 to 12 and FIGS. 1 to 15.

FIG. 1 represents a scheme of synthesis of protein-functionalized gold nanoparticles. 1—Synthesis of bare gold nanoparticles; 2—Functionalization and steric stabilization of gold nanoparticles by hetero-bifunctional PEO-1 layer; 3—Functionalization by hetero-bifunctional PEO-2 layer with surface-exposed SH-reactive groups; 4—Bio-functionalization with Annexin-A5-SH or Annexin-A5-ZZ-SH proteins, or any other protein exposing SH groups. Oriented binding of antibodies to the ZZ fragment.

FIG. 2 illustrates the synthesis of gold nanoparticles functionalized with Annexin-A5-SH protein. The stereo-specific insertion of the cysteine residue on a solvent-exposed loop at the concave face of Annexin-A5, which is opposed to the site of binding to PS-containing membranes ensures maximum efficiency of binding.

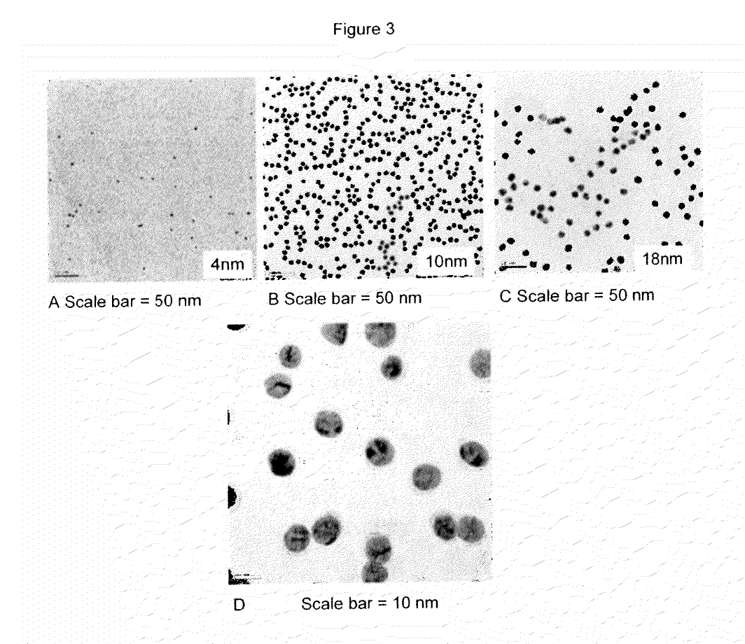

FIG. 3 represents Transmission Electron Microscopy (TEM) images of gold nanoparticles of different sizes according to the instant invention. A,B,C: 4, 10 and 18 nm-diameter gold nanoparticles prepared according to example 1.1 (scale bar=50 nm). D: gold nanoparticles of solAPP-A5 prepared according to example 3 (scale bar=10 nm).

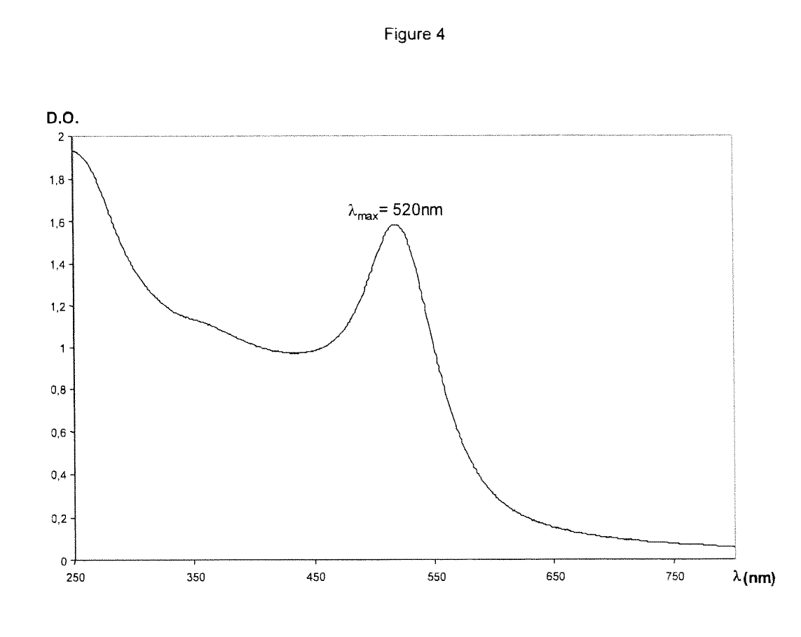

FIG. 4 represents the UV-visible absorption spectrum of a sol of bare gold nanoparticles of 10 nm-diameter prepared according to example 1.1. The absorption band at 520 nm is due to plasmon resonance of the surface gold atoms.

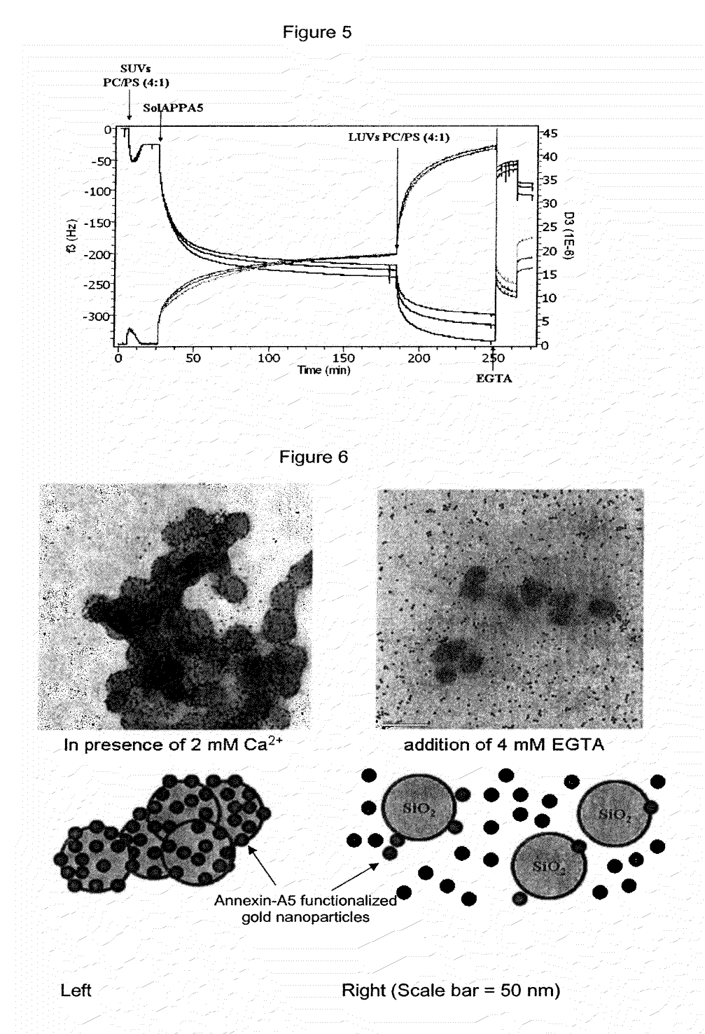

FIG. 5 represents Quartz Crystal Microbalance with Dissipation monitoring (QCM-D) measurements of the binding of Anx5-functionalized gold nanoparticles (solAPP-A5) prepared according to example 3 to a 1,2-dioleyl-sn-glycero-3-phosphatidylcholine/1,2-dioleyl-sn-glycero-3-phosphatidylserine (PC/PS) (molar ratio 4:1) supported lipid bilayer, followed by the binding of (PC/PS) (4:1) large unilamellar liposomes (LUVs) to the monolayer of nanoparticles.

FIG. 6 represents TEM images (top row) and a scheme (bottom row) of the specific interaction of functionalized gold nanoparticles (solAPP-A5) prepared according to example 3 with silica particles coated with supported lipid bilayers containing phosphatidylserine, in the presence of calcium (left), and their re-dispersion in the presence of EGTA, a calcium chelating agent (right) (scale bar=200 nm).

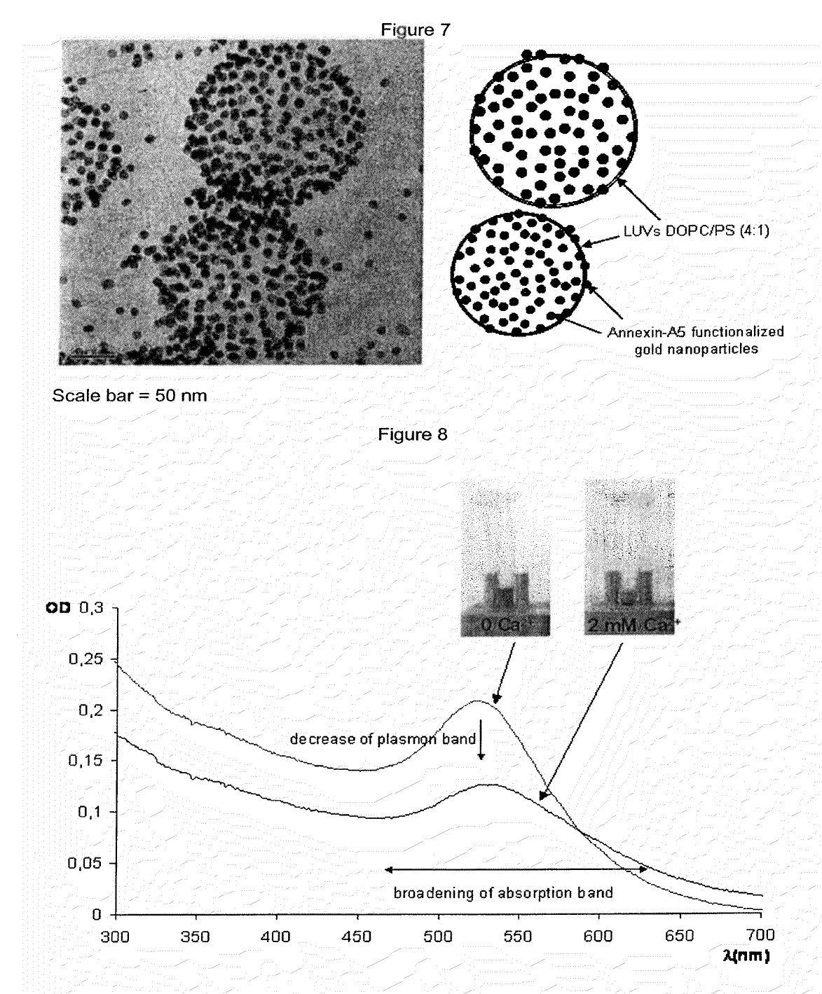

FIG. 7 represents a cryo-TEM image showing the high-density binding of gold nanoparticles from the solAPP-A5 prepared according to example 3 to large unilamellar liposomes (LUVs) containing phosphatidylserine, in the presence of calcium (scale bar=50 nm).

FIG. 8 represents a UV-visible absorption spectrum of gold nanoparticles from the solAPP-A5 prepared according to example 3 in the presence of silica particles coated with supported lipid bilayers containing phosphatidylserine in the presence (blue) and in the absence (red) of calcium.

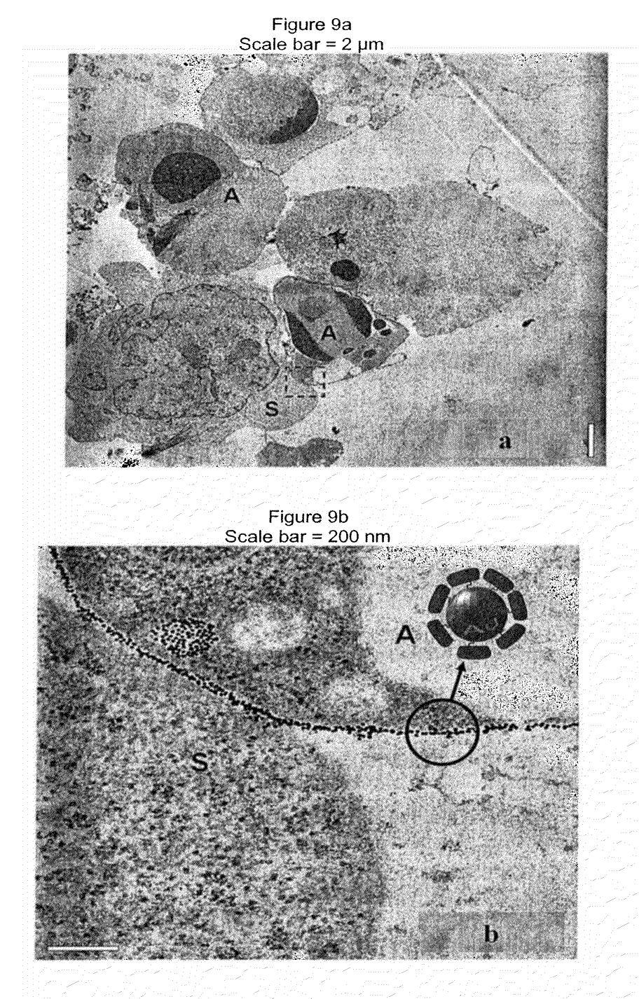

FIG. 9 illustrates the application of Anx5-functionalized gold nanoparticles according to the instant invention for labelling cell membranes exposing phosphatidylserine molecules. TEM images of apoptotic bodies labelled with gold nanoparticles of the solAPP-A5 prepared according to example 3. FIG. 9a shows a healthy cell (S) and apoptotic bodies (A) with characteristic domains of condensed chromatin (scale bar=2 μm). FIG. 9b shows a high-magnification image of the area marked with dotted lines in FIG. 9a, where a healthy cell and an apoptotic body are adjacent (scale bar=200 nm). Anx5-coupled gold nanoparticles cover entirely the apoptotic body, at maximal density, while the healthy cell shows no labelling.

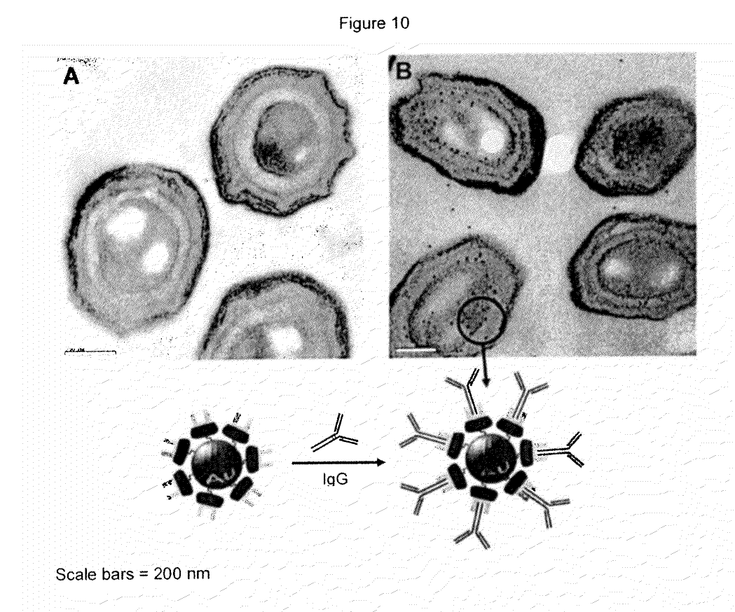

FIG. 10 illustrates the application of Anx5-ZZ-functionalized gold nanoparticles for labelling cellular antigens. Labelling of antigens from Bacillus subtilis spores with gold-Anx5-ZZ nanoparticles specifically-bound to an anti-spore IgG. A—Control experiment, in which spore sections were incubated with gold nanoparticles functionalized with Anx5-ZZ fusion proteins in the absence of anti-spore antibody. Not a single gold particle is visible on this section; B—Labelling of spores with anti-spore antibody followed by specific binding of gold-Anx5-ZZ nanoparticles. Gold nanoparticles label at high density a region of the spore corresponding to the periphery of the core domain. (scale bars=200 nm).

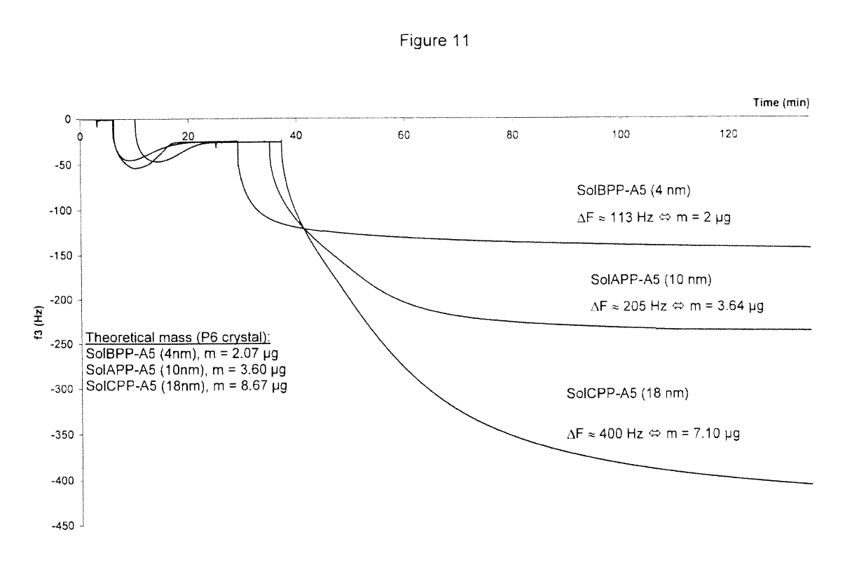

FIG. 11 represents QCM-D measurements of the binding of Anx5-functionalized gold nanoparticles of solAPP-A5, solBPP-A5 and solCPP-A5, prepared according to example 10, to a PC/PS (4:1) supported lipid bilayer.

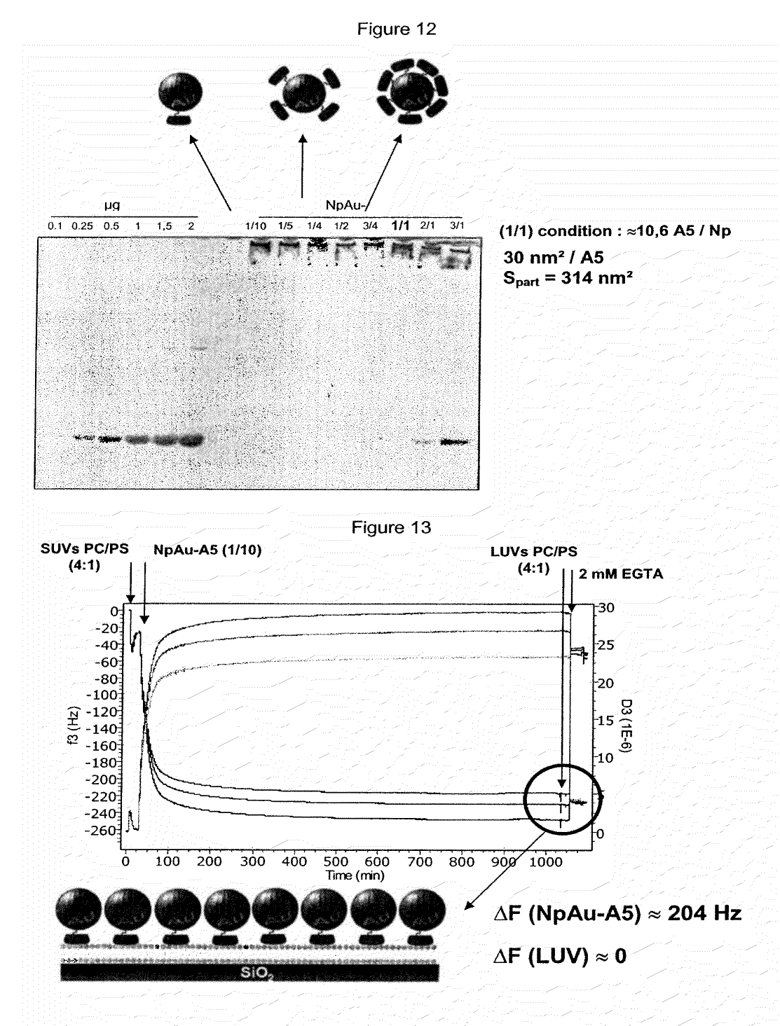

FIG. 12 represents results of a polyacrylamide gel electrophoresis (PAGE) in denaturing conditions (in presence of sodium dodecyl sulphate) allowing to measure the amount of Anx5 which can be covalently coupled to gold nanoparticles of solAPPmal.

FIG. 13 represents QCM-D measurements of the binding of Anx5-functionalized gold nanoparticles of solAPP-A5 in 1/10 condition (1 Anx5 molecule per gold nanoparticle) prepared according to example 11 to a PC/PS (4:1) supported lipid bilayer.

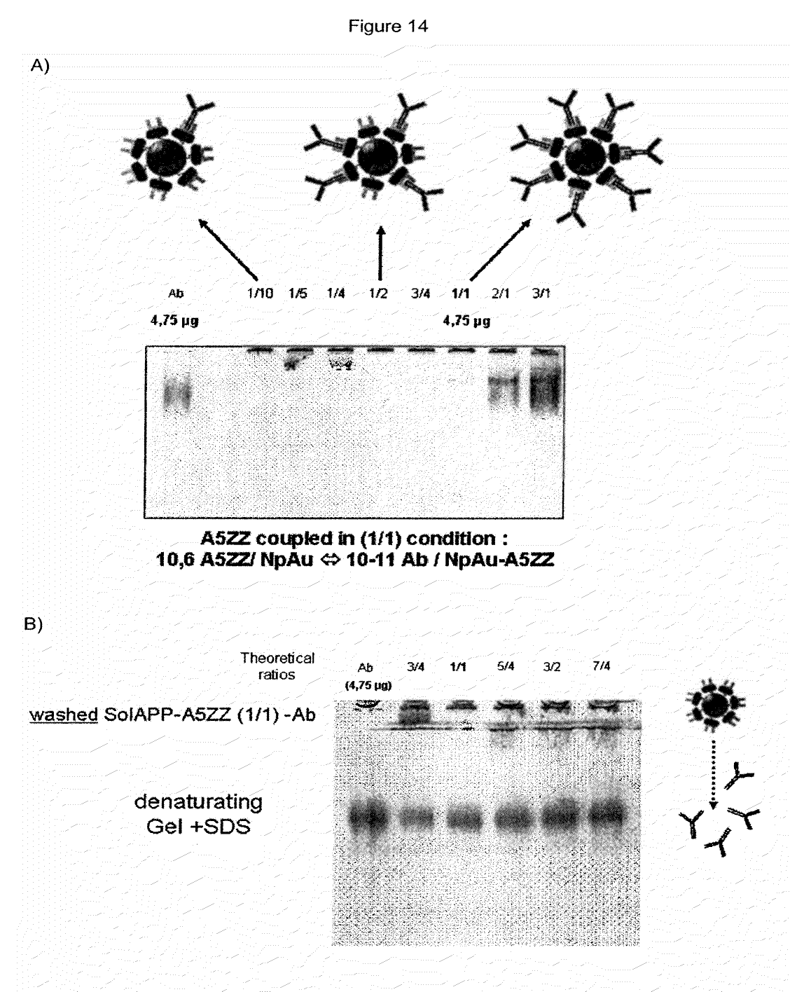

FIG. 14 represents results of a polyacrylamide gel electrophoresis (PAGE) in non-denaturing (A) and denaturing (B) conditions allowing to measure the maximum amount of antibodies (anti-PY79 spores) which can be bound to gold nanoparticles of solAPP-A5-ZZ prepared in 1/1 condition (saturating condition) following the procedure described in example 12.

FIG. 15 represents a model of a side-view of annexin-A5 (Anx5) bound to a PS-containing lipid membrane surface. The Anx5 molecule is a slightly curved shape, with a convex membrane-binding face and a concave face opposite to the membrane-binding face. Arrow 1 points to the position of a solvent-exposed loop on the concave face of Anx5, which contains the sequence T163, A164, 1165. The replacement of one of these amino-acids by a cysteine creates a —SH group in a highly accessible position, allowing the subsequent coupling of gold nanoparticles functionalized with a spacer ending with a SH-reactive group. The C-terminus of Anx5 is located close to the concave face of Anx5, in a slightly buried position. Fusion proteins between the C-terminal end of Anx5 and the N-terminal end of any protein or protein domain will position said protein or protein fragment close the concave face. This is illustrated in the case of the ZZ domain of protein A from Staphylococcus aureus. The dashed line represents the polypeptide linking Anx5 to the ZZ domain. Arrow 2 points to a loop which is highly exposed when the protein is not bound to the membrane. This loop contains the sequence G259, A260, G261. Other loops located on the concave face of Anx5 contain sequences G28, L29, G30, G100, A101, G102, W185, G186, T187. The replacement of one of these amino-acids in Anx5-Z fusion protein or Anx5-ZZ fusion protein by a cysteine creates a —SH group in a highly accessible position, allowing the subsequent coupling of gold nanoparticles functionalized with a spacer ending with a SH-reactive group.

### EXAMPLE 1

### Synthesis of Aqueous Suspensions of 10 nm-Diameter Gold Nanoparticles, Functionalized Covalently and Stereo-Specifically with Proteins

1.1 Preparation of 10 nm-Diameter Gold Nanoparticles (solA).

Gold nanoparticles are prepared according to a method derived from the protocol of Turkevich et al. (25) in which tetrachloroaurate salts (HAuCl₄, KAuCl₄) are reduced by citrates, leading to the formation of suspensions (called sol hereunder) of 10 nm-diameter gold nanoparticles.

Typically, for preparing 550 mL of aqueous sol of gold nanoparticles of 10 nm-diameter (FIG. 33) and for a concentration equal to 32.62 nM of particles (1.964.10¹⁶ particles/L, namely 10⁻³ M Au): a volume of 400 mL of ultrapure water (<18 MΩ, system of purification Millipore Synergy, Simpak®1) is carried to boiling. 100 mL of an aqueous solution of 0.55 M KAuCl₄ (99.999%, Aldrich) are added. The reaction medium is carried to the water reflux (110° C.). The reduction of auric salts occurs upon addition of 50 mL of 3.4 mM sodium citrate dihydrate solution (99%, Aldrich). The reaction is left 30 minutes to the water reflux and then cooled at room temperature.

### 1.2 Functionalization of the Gold Nanoparticles and Steric Colloidal Stabilization.

The coupling of hetero-bifunctional PEO macromolecules bearing a thiol (—SH) group in w position and amine (—NH₂) group in α position is carried out in two steps. The thiol group allows their covalent coupling with the formation of Au—S bonds with the surface gold sites. The presence of amine groups allows the subsequent coupling to molecules of interest. First, a homo-bifunctional bis-amino telechelic PEO is modified by thiolation (addition of thiol) of primary amines by 2-iminothiolane, and second the thiolated macromolecules are coupled to the surface of the nanoparticles.

### 1.2.1 Thiolation of Bis-Amino Telechelic PEO Macromolecules by 2-Iminothiolane.

In a 50 mL beaker, 1 g of bis-amino telechelic PEO (M_{w}=1628 g/mol, Aldrich) is dissolved in 20 mL of borate buffer composed of 0.1 M boric acid (99%, Sigma), mM of ethylenediaminetetraacetic acid (EDTA, 99.6%, Sigma) and adjusted at pH 8 with NaOH. After complete dissolution of the polymer, 1 mL of an aqueous solution of 2-iminothiolane (98%, Aldrich) of concentration equal to 0.614 mol/L is added. The mixture is left reacting for at least 4 hours.

1.2.2 Coupling of α-amino-ω-mercapto-PEO macromolecules to gold nanoparticles (Nu₁-PEO-Nu₂ with Nu₁=HS and Nu₂=NH₂).

After 4 hours of incubation, 232 mg of sodium borohydride are added in order to prevent the formation of disulfide bonds. The pH is adjusted to 6.5-7 with HCl. After 15 minutes of agitation (end of the gaseous emission), the modified polymer is transferred to a 500 mL beaker.

Then, 333 mL of solA of gold nanoparticles obtained in example 1.1. are added in this medium under strong stirring. The number of moles of macromolecules corresponds to 12 times the equivalent number of moles of surface gold atoms. This excess allows the saturation of the surface by polymer molecules and at the same time prevents cyclization phenomena of the macromolecules on a same nanoparticle or a bridging between several nanoparticles. The nanoparticles are incubated in the presence of polymer for at least 12 hours.

### 1.2.3 Purification of Functionalized Gold Nanoparticles.

The objective of this operation is to eliminate the excess of hetero-bifunctional PEOs and to concentrate the sol of modified gold nanoparticles in a minimal volume (<mL) for a particle concentration higher than 0.1 μM, typically equal to 0.834 μM (5.02.10¹⁷ particles/L), in order to increase the rates of reaction on the surfaces for the next coupling steps. The volume of dispersion is reduced to 10 mL by water evaporation under reduced pressure at 70° C. using a rotary evaporator. The elimination of the polymer excess can be accomplished either by ultrafiltration (Amicon®, Millipore) using regenerated cellulose membrane with a cut-off threshold of 100 kDa under nitrogen pressure, by centrifugation with Microcon® or Centricon® (Millipore) ultrafiltration systems, or by ultracentrifugation (25,000 rpm namely 34,000 g, 15 min, 4° C.) using an ultracentrifuge (Optima™ of Beckman Coulter™). The latter method is preferred because it allows eliminating the aggregates formed during coupling, due to gradients of concentration generated during the addition of solA in the reaction medium. In both cases several cycles of washing with ultrapure water have to be carried out so that the maximum residual polymer concentration does not exceed mol/L. Purification on exclusion column of Sephadex® type is also possible.

The sol of 10 nm-diameter gold nanoparticles modified by α-amino-ω-mercapto-poly(ethylene oxide) (M_{w}=1737 g/mol) is named solAPN hereafter. The number of α-amino-ω-mercapto-poly(ethylene oxide) molecule per gold nanoparticle has been determined by measuring the thiol groups with the Elmann reagent (5,5′-dithio-bis-(2-nitrobenzoic acid) after reduction of the nanoparticles of solAPN. The number of macromolecules per gold nanoparticles is equal to 1070 which gives a molecular surface coverage of 0.29 nm², value close to those found for self assembled monolayers of dodecane thiolate (32) on gold surface (0.21 nm²) and thiolated poly(ethylene glycol) with higher molecular weight (0.35 nm², for a Mw=5000 g/mol).

1.3 Coupling of Hetero-Bifunctional PEO (NHS-PEG-Mal) On the Surface of the Modified Gold Nanoparticles of solAPN

This step aims at saturating the surface of gold nanoparticles with maleimide groups, which are able to react specifically with thiols of cysteine residues present in certain proteins, peptides, or other molecules. The conditions of coupling are chosen for maintaining the colloidal stability of the nanoparticles. The hetero-bifunctional PEO NHS-PEG-Mal (M=3400 g/mol, 85%, Nektar, the USA) allows to carry out this step because the PEO chain is sufficiently hydrophilic to preserve the solubility of the particles. The coupling is carried out by nucleophilic substitution (SN₂) of ester of N-hydroxysuccinimide (NHS) by the primary amines ending the chains of PEO grafted on the nanoparticles of the solAPN, leading to the formation of an amide bridge between the two macromolecules. The resulting sol is called solAPPmal hereafter.

A 1 mL volume of solAPN of concentration equal to 0.834 μM of gold nanoparticles prepared according to step 1.2.3 is diluted in 1 mL of N-(2-Hydroxyethyl)piperazine-N′-(2-ethanesulfonic acid) (HEPES, Sigma) or phosphate buffer of 100 mM concentration at pH 7. A mass of 31.7 mg of NHS-PEG-Mal powder is directly added to the solution under vigorous stirring (1,200 rpm with the vortex) until complete dissolution of the polymer. The quantity of NHS-PEG-Mal to be added is calculated from the number of moles of surface gold sites (nAuₛ=3.96 μmoles), considering 100% coverage of the nanoparticle surface by the ethylene α-amino-ω-mercapto-PEO and by applying a twofold excess compared to this number of sites. The reaction is left reacting for 2 hours at room temperature, under low stirring. The determining parameter of this step is the reaction kinetics of esters of N-hydroxysuccinimide with the amines of the nanoparticle surface of the solAPN, optimized by the use of a high concentration of nanoparticles, compared to the kinetics of hydrolysis of the maleimide groups which is reduced at neutral pH.

The purification of the nanoparticles is carried out by centrifugation or by ultrafiltration according to the protocol described in 1.2.3. After elimination of the supernatant, the pellet is re-dispersed in 50 mM HEPES or phosphate buffer, 10 mM EDTA, adjusted to pH 7, degassed under vacuum and covered with argon. Several cycles of washing are carried out so that the residual concentration of NHS-PEG-Mal in the solAPPmal is lower than 10⁻⁸ M. The volume of the solAPPmal is brought back to 200 μL for a concentration of 4.17 μM of gold nanoparticles. The number of maleimido groups per gold nanoparticle has been determined by reaction with the Elmann reagent (5,5′-dithio-bis-(2-nitrobenzoic acid) reduced by the tris(2-carboxyethyl)phosphine). The number of maleido groups per gold nanoparticle of the solAPPmal is equal to 1000. This value is close to the number of thiolated macromolecules per gold nanoparticles previously found which demonstrate that all the amino groups have reacted with the N-hydroxysuccinimide esters.

1.4. Coupling of Anx5-SH to the Functionalized Gold Nanoparticles of solAPPmal.

The coupling of SH-exposing proteins to gold nanoparticles of solAPPmal was achieved using the double mutant Annexin-A5 [T163C; C314S] which presents a unique SH group, as described in the patent application WO2005114192 (22). Anx5-SH monomer is obtained by reduction of Anx5-S—S-Anx5 dimers by dithiothreitol (DTT, Sigma) and purification by anion exchange chromatography.

To 900 μL of Anx5-S—S-Anx5 at 1.88 mg/mL in 20 mM 2-Amino-2-(hydroxymethyl)-1,3-propanediol (Tris, Sigma) buffer, pH 8, 0.02% NaN₃, 100 μL of 0.1 M DTT are added. The medium is left reacting for 1h40 at room temperature. The protein is purified on a MonoQ HR5/5 column (Amersham Biosciences) pre-equilibrated with 50 mM HEPES or phosphate, pH 7, 10 mM EDTA, degassed under vacuum and covered with argon. A sample of 1 mL of Anx5-SH at 0.94 mg/mL is pooled into a microtube “weak adhesion” (Simport, Canada).

A volume of 119 μL of solAPPmal of concentration equal to 4.17 μM of particles prepared according to example 1.3 is added to the Anx5-SH solution under stirring with a vortex. The tube is closed under inert atmosphere (argon) and the reaction is left for at least 12 h at room temperature. The quantity of Anx5 used for the coupling corresponds to 5 times the theoretical quantity of annexin necessary to cover totally the surface developed by the gold nanoparticles. In this step the critical parameter is the kinetics of alkylation of thiols by the maleimides which must be privileged compared to the concurrent reactions which are the hydrolysis of the maleimides and the oxydation of thiols. Considering the low numbers of moles used (2.63×10⁻⁸ mole of thiol and 2.3×10⁻⁶ mole (theoretical) of maleimide), it is thus necessary to increase the concentration in gold nanoparticles to the maximum and to degas the solutions in order to eliminate dissolved dioxygen. The resulting suspension of Anx5-functionalized gold nanoparticles is called solAPP-A5 hereafter.

Purification is carried out by centrifugation or by ultrafiltration according to the protocol described in example 1.2.3. After elimination of the supernatant, the pellet is re-dispersed in 10 mM HEPES, pH 7.4, 150 mM NaCl, 2 mM NaN₃. After 4 cycles of washing, solAPP-A5 particles of concentration equal to 1.75 μM of particles (namely 1.056.10¹⁸ particles/L) are diluted to 0.234 μM of particles (1.408.10¹⁷ particles/L) in a volume of 1.5 mL of the same buffer and are stored in weak adhesion microtubes at 4° C. This sol is stable in physiological medium, in the presence of calcium ions and does not present any sign of flocculation (i.e. colloidal destabilization) after several months of storage.

TEM images of solAPP-A5 particles (FIG. 3 D) show an additional density (thickness equal to about 4 nm) around the gold nanoparticles, attesting the presence of coupled proteins.

The same protocol is used for coupling Anx5-ZZ molecules to functionalized gold nanoparticles of solAPPmal. Two mutant Anx5 molecules were used: [T1630; C314S] and [A260C; C314S], leading to almost identical results.

### EXAMPLE 2

### UV-Visible Absorption Spectroscopy of Suspensions of Gold Nanoparticles

Measurements of UV-visible absorption spectra of gold nanoparticle suspensions give access to the concentration of gold nanoparticles of SolA, SolAPN, SolAPPmal, SolAPP-A5 and to the quality of these dispersions with respect to their colloidal stability.

The UV-visible spectrum of 10 nm-diameter gold nanoparticles presents an absorption band at a wavelength λ=520 nm attributed to the plasmon resonance band of gold nanoparticles (FIG. 4). For a given particle size, the optical density is directly proportional to the particle concentration, which is determined by the Beer-Lambert law: O.D.=εₚ.Cₚ.l, where O.D. is the optical density, εₚ the molar extinction coefficient of 10 nm-diameter gold particles (εₚ=1.086×10⁸ (mole of particules)⁻¹.L·cm), Cₚ the concentration in mole of particles, L the path length (1 cm).

### EXAMPLE 3

### Binding of solAPP-A5 Gold Nanoparticles to Supported Lipid Bilayers, by Quartz Crystal Microbalance with Dissipation Monitoring (QCM-D)

The binding of Anx5-functionalized gold nanoparticles (solAPP-A5), prepared according to example 1.4, to supported lipid bilayers containing PS was measured in a quantitative manner by the QCM-D method (37), according to reference measurements established for Anx5 (17).

FIG. 5 shows 1) that the kinetics of binding of the solAPP-A5 particles (blue curve) to a (PC/PS, 4:1) supported lipid bilayers saturates, 2) that Anx5-gold nanoparticles bound to a supported lipid bilayer are able to bind PS-containing liposomes, demonstrating that several molecules of Anx5 are bound per solAPP-A5 nanoparticle, 3) that the binding of solAPP-A5 particles is PS-specific, as their binding is only reversed by addition of the calcium chelating agent ethylene-bis(oxyethylenenitrilo)tetraacetic acid (EGTA).

The mass of Anx5-coupled gold nanoparticles bound to the supported lipid bilayer at saturation is close to 3.64 μg, as determined from the Sauerbrey equation (38) (which states that the adsorbed mass is proportional to the variation in frequency ΔF with m=−C.ΔF (with C=17.7 ng/cm²)). This value is almost equal to the theoretical mass calculated (3.6 μg), assuming that the nanoparticles form a close-packed 2D assembly of nanoparticles.

### EXAMPLE 4

### Assay for Assessing the Calcium-Dependent Binding of solAPP-A5 Gold Nanoparticles to Supported Lipid Bilayers Containing Phosphatidylserine

A simple and rapid macroscopic assay has been developed to evaluate the property of solAPP-A5 nanoparticles to bind in a calcium-dependent manner to PS-containing supported lipid bilayers deposited around silica particles, referred to as nanoSLBs (39). The binding of solAPP-A5 nanoparticles to nanoSLBs induces the flocculation of the silica particles, which is accompanied by a pink-to-blue colour change visible to the naked eye (FIG. 6-left). Conversely, the addition of the calcium chelating agent EGTA induces the re-dispersion of the gold nanoparticles (FIG. 6-right).

The nanoSLBs were prepared according to the protocol previously described (39). In a microtube Eppendorf® containing 10 μL of 10 mM HEPES, pH 7.4, 150 mM NaCl, 2 mM CaCl₂, 5 μL of nanoSLBs of concentration equal to 5 mg/mL of silica particles are added. A volume of 2 μL of 20 mM CaCl₂ is added to get a final Ca²⁺ concentration of mM. A volume of 5 μL of solAPP-A5 at 0.234 μM of particles prepared according to example 1.4 is then added in the medium. The particles flocculate instantaneously and sediment quickly. For more diluted concentrations of NpAu-A5 (10 to 100 times), flocculation is visible through a change of colour, from pink to blue, followed by sedimentation and adsorption of the particle aggregates on the walls of the tube.

The realization of this test in the absence of calcium does not lead to the flocculation of the nanoparticles. In the same way, the addition of 1.76 μL of 50 mM EGTA causes the instantaneous re-dispersion of the NpAu-A5 gold nanoparticles.

### EXAMPLE 5

### Specific Binding of solAPP-A5 gold Nanoparticles to Large Unilamellar Liposomes (LUV) Containing PS Molecules in the Presence of Calcium, Revealed by Cryo-TEM

FIG. 7 shows solAPP-A5 nanoparticles bound to the surface of PS-containing LUVs, by cryo-TEM (40).

150 nm-diameter LUVs made of 1,2-dioleyl-sn-glycero-3-phosphocholine/1,2-dioleyl-sn-glycero-3-phosphoserine (DOPC/DOPS) (4:1) are prepared by standard phase reversion procedures. A dispersion of 50 μg/mL LUVs is prepared in a buffered solution of 10 mM HEPES, pH 7.4, 150 mM NaCl and 4 mM CaCl₂. A volume of 11 μL of 0.264 μM solAPP-A5 particles prepared according to example 1.4 is added to a volume of 11 μl of LUV. 2 μL of the mixyute are deposited on a perforated carbon EM grid, the excess of liquid is blotted with a filter paper and the thin liquid film is quickly frozen by plunging the grid into nitrogen-cooled liquid ethane (40). Cryo-TEM is performed with a Tecnai F20 microscope (FEI) operating at 200 kV.

The LUV surface is entirely covered with Anx5-functionalized gold nanoparticles.

### EXAMPLE 6

### Binding of solAPP-A5 Gold Nanoparticles to Supported Lipid Bilayers on Silica Nanoparticles (nanoSLBs) by UV-Visible Spectroscopy

The specific calcium-dependent binding of solAPP-A5 gold nanoparticles to PS-containing nanoSLBs can be measured by UV-visible spectroscopy. In the absence of calcium ions, stable sols are observed (FIG. 8-red curve). Upon addition of calcium, the sols become unstable due to the formation of aggregates, as observed by TEM (FIG. 6-left). Unstable gold nanoparticles suspensions show several characteristic features by UV-visible spectroscopy (FIG. 8-blue curve): the absorption band is shifted towards larger wavelength; the spectrum presents a broadening, with increase of the half-maximum width and decrease of the maximal OD value.

### EXAMPLE 7

### Labelling of Apoptotic Bodies of BCR-ABL Cells with solAPP-A5 Gold Nanoparticles, by TEM

Chronic myeloid leukaemia (CML) is characterized by a genetic defect associated with a chromosomal translocation between chromosomes 9 and 22, the molecular consequence of which is the synthesis of a chimerical protein, called BCR-ABL, having a constitutive tyrosine kinase activity inducing the incapacity of the BCR-ABL cells to enter into apoptosis. Apoptosis can be induced in BCR-ABL cells by treatment with STI-571 (Gleevec, Novartis), a compound inhibiting tyrosine kinases of ABL type (41).

The solAPP-A5 gold particles are used to follow the process of STI-571-induced apoptosis in BCR-ABL cells, using the classical method of ultramicrotomy followed by TEM observation (42).

Typically, 2×10⁵ BCR-ABL cells in 500 μL of culture medium are treated with 1 μM STI-571 incubated at 37° C. in the presence of 5% CO₂ for various time periods, after which the excess of STI-571 is removed by three cycles of sedimentation at 1,000 rpm for 10 min followed by re-suspension into 500 μL of a buffer made of 150 mM NaCl and 10 mM HEPES, pH 7.4, for the two first cycles. After the third cycle of sedimentation, the cells are re-suspended with 320 μL of a buffer made of 150 mM NaCl, 2 mM Ca²⁺′ 10 mM HEPES, pH 7.4, to which 180 μL of solAPP-A5 containing 1.4×10¹⁵ particles/L are added. After 1 hr of incubation at about 20° C., the excess of nanoparticles is removed by 3 cycles of centrifugation at 1,000 rpm followed by re-suspension in 500 μL of a buffer made of 150 mM NaCl, 2 mM Ca²⁺′ 10 mM HEPES, pH 7.4. The cells are then fixed in the presence of 2.5% glutaraldehyde/4% paraformaldehyde for overnight, rinsed in cacodylate 0.2M, fixed with 1% OsO₄, rinsed in cacodylate 0.2M, dehydrated in successive baths of increasing concentrations of ethanol and embedded in an epoxy resin, according to the protocols commonly used in the field (42). Ultrathin sections (65 nm-thickness) are made from the cell pellets. The sections are stained with 5% uranyl acetate for 10 min and observed by TEM.

FIG. 9a, corresponding to a 18 h treatment in the presence of 1 μM STI-571, shows a healthy cell (S) together with apoptotic bodies (A) presenting characteristic domains of condensed chromatin.

FIG. 9b shows an enlarged view with adjacent areas from a healthy cell and an apoptotic body. Anx5-coupled gold nanoparticles cover entirely the membrane of the apoptotic body, at maximal density, while the healthy cell is entirely devoid of gold particles. The images shown here are representative of the whole sample.

These images demonstrate that labelling is specific and reaches a high surface density. The labelled apoptotic membranes are detectable both by TEM and by optical microscopy.

The specificity and intensity of the labelling have allowed a detailed analysis of the kinetics of the apoptotic process. Apoptotic bodies are observed, in low number, after only 1 hour of treatment with STI-571. The number of apoptotic bodies increases with the time of treatment and approximately 50% of the cells are in apoptosis after 48 hours of treatment.

### EXAMPLE 8

### Immuno-Labelling of Spore Antigens from Bacillus subtilis with Gold-Anx5-ZZ Nanoparticles Anchoring Anti-Spore Antibodies

The property of the ZZ fragment of protein A from Staphylococcus aureus (23, 24) to bind to the Fc fragment of IgGs provides to gold nanoparticles functionalized covalently and stereo-selectively with Anx5-ZZ-SH the capacity of a generic platform to label cellular antigens via specific antibodies. The proof of concept is developed for labelling surface antigens from Bacillus subtilis spores with an anti-spore IgG.

Bacillus subtilis spores are processed for ultramicrotomy according to standard procedures (42). The thin sections, supported on a carbon film deposited on an electron microscopy grid, are placed on top of a 17-μL drop of phosphate saline buffer (PBS) containing 1% BSA, for 1 hr at about 20° C. The grid is transferred on top of a 17-μL drop containing 5 μg/mL anti-spore polyclonal antibodies in PBS-0.2% BSA for 1 hr at about 20° C., after which three steps of rinsing are performed to remove unbound antibodies by transferring the grid successively on top of PBS-0.2% BSA drops. The grid is then transferred to a drop containing 1.4×10¹⁵ particles/L Anx5-ZZ-coupled gold nanoparticles in PBS-0.2% BSA for 30 min at about 20° C., after which three steps of rinsing are performed by transferring the grid successively on top of PBS-0.2% BSA drops. The section is then fixed with 2.5% glutaraldehyde/4% paraformaldehyde in 0.2 M cacodylate pH 7.2 for 2 min, rinsed with water, and finally stained with 5% uranyl acetate for 10 min.

FIG. 10B shows gold particles labelling a specific area of the spore corresponding to the periphery of the core domain (43).

The specificity of labelling is demonstrated by FIG. 10A which shows a sample in which the step of incubation in the presence of anti-spore antibodies has been omitted before addition of the Anx5-ZZ-coupled gold nanoparticles. Not a single gold particle is visible on the section.

### EXAMPLE 9

### Extension of the Procedure of Synthesis of 10 nm-Gold Nanoparticles Functionalized Covalently and Stereo-Specifically with Proteins to Gold Nanoparticles with Different Sizes

9.1 Preparation of 4 nm-Diameter Gold Nanoparticles (solB).

4 nm gold nanoparticles are prepared according to the method derived from the protocol of Murphy et al. (44) in which tetrachloroaurate salts (HAuCl₄, KAuCl₄) are reduced by sodium borohydride in presence of sodium citrate, leading to the formation of sols of 4 nm-diameter gold nanoparticles.

Typically, for preparing 103 mL of aqueous sol of gold nanoparticles of 4 nm-diameter (FIG. 3A) and for a concentration equal to 122.8 nM of particles (7,393.10¹⁶ particles/L, namely 2.43 10⁻⁴ M Au): a volume of 100 mL of an aqueous solution of 2.5 10⁻⁴ M of HAuCl₄ (99.999%, Aldrich) and 2.5 10⁻⁴ M of sodium citrate tribasic dehydrate (>99.0%, Fluka) is prepared with ultrapure water (<18 MΩ, system of purification Millipore Synergy, Simpak®1). 3 mL of an aqueous solution of 0.1 M NaBH₄ (99%, Aldrich) are added under strong stirring. The reduction of auric salts spontaneously occurs upon addition of the reducer agent. The reaction is left 10 minutes under stirring and then let at room temperature.

9.2 Preparation of 18 nm-Diameter Gold Nanoparticles (solC).

Gold nanoparticles are prepared according to a variation of the protocol of Frens et al. (26) in which tetrachloroaurate salts (HAuCl₄, KAuCl₄) are reduced by citrates, leading to the formation of sols of 18 nm-diameter gold nanoparticles.

Typically, for preparing 360 mL of aqueous sol of gold nanoparticles of 18 nm-diameter (FIG. 3C) and for a concentration equal to 1.54 nM of particles (9.285.10¹⁴ particles/L, namely 2.78 10⁻⁴ M Au): a volume of 350 mL of ultrapure water (<18 MΩ, system of purification Millipore Synergy, Simpak®1) is carried to boiling. 50 mL of an aqueous solution of 2 10⁻³ M HAuCl₄ (99.999%, Aldrich) are added. The reaction medium is carried to the water reflux (110° C.). The reduction of auric salts occurs upon addition of 40 mL of 1% w/w sodium citrate dihydrate solution (99%, Aldrich). The reaction is left 30 minutes to the water boiling in order to concentrate the solution until a volume equal to 360 mL and then cooled at room temperature.

9.3 Functionalization of the Gold Nanoparticles and Steric Colloidal Stabilization.

The coupling of hetero-bifunctional PEO macromolecules bearing a thiol (—SH) group in ω position and amine (—NH2) group in α position is carried out with the same procedure described in §1.2.

9.3.1 Coupling of α-amino-ω-mercapto-PEO Macromolecules to Gold Nanoparticles of Sol B (4 nm Gold Nanoparticles) (Nu₁-PEO-Nu₂ with Nu₁=HS and Nu₂=NH₂).

In a 50 mL beaker, 1 g of bis-amino telechelic PEO (M_{w}=1628 g/mol, Aldrich) is dissolved in 20 mL of borate buffer composed of 0.1 M boric acid (99%, Sigma), mM of ethylenediaminetetraacetic acid (EDTA, 99.6%, Sigma) and adjusted at pH 8 with NaOH. After complete dissolution of the polymer, 1 mL of an aqueous solution of 2-iminothiolane (98%, Aldrich) of concentration equal to 0.614 mol/L is added. The mixture is left reacting for at least 4 hours.

After 4 hours of incubation, 232 mg of sodium borohydride are added in order to prevent the formation of disulfide bonds. The pH is adjusted to 6.5-7 with HCl. After 15 minutes of agitation (end of the gaseous emission), 18.2 mL of the modified polymer is transferred to a 250 mL beaker.

Then, 103 mL of solB of gold nanoparticles obtained in example 9.1. are added to this medium under strong stirring. The number of moles of macromolecules corresponds to 12 times the equivalent number of moles of surface gold atoms. The nanoparticles are incubated in the presence of polymer for at least 12 hours.

9.3.2 Purification of Functionalized 4 nm Gold Nanoparticles.

The objective of this operation is to eliminate the excess of hetero-bifunctional PEOs and to concentrate the sol of modified gold nanoparticles in a minimal volume (<mL) for a particle concentration higher than 1 μM, typically equal to 3.12 μM (1.878.10¹⁸ particles/L), in order to increase the rates of reaction on the surfaces for the next couplings. The volume of dispersion is reduced to 10 mL by water evaporation under reduced pressure at 70° C. using a rotary evaporator. The elimination of the polymer excess can be accomplished by ultracentrifugation (80,000 rpm, 15 min, 4° C.) using an ultracentrifuge (Optima™ of Beckman Coulter™). Several cycles of washing with ultrapure water have to be carried out so that the maximum residual polymer concentration does not exceed 10⁻⁷ mol/L. Purification on exclusion column of Sephadex® type is also possible at this step.

The sol of 4 nm-diameter gold nanoparticles modified by α-amino-ω-mercapto-poly(ethylene oxide) (M_{w}=1737 g/mol) is named solBPN hereafter.

9.3.3 Coupling of α-amino-ω-mercapto-PEO Macromolecules to Gold Nanoparticles of Sol C (18 nm Gold Nanoparticles) (Nu₁-PEO-Nu₂ with Nu₁=HS and Nu₂=NH₂).

In a 50 mL beaker, 1 g of bis-amino telechelic PEO (M_{w}=1628 g/mol, Aldrich) is dissolved in 20 mL of borate buffer composed of 0.1 M boric acid (99%, Sigma), mM of ethylenediaminetetraacetic acid (EDTA, 99.6%, Sigma) and adjusted at pH 8 with NaOH. After complete dissolution of the polymer, 1 mL of an aqueous solution of 2-iminothiolane (98%, Aldrich) of concentration equal to 0.614 mol/L is added. The mixture is left reacting for at least 4 hours.

After 4 hours of incubation, 232 mg of sodium borohydride are added in order to prevent the formation of disulphide bonds. The pH is adjusted to 6.5-7 with HCl. After 15 minutes of agitation (end of the gaseous emission), 16.2 mL of the modified polymer is transferred to a 500 mL beaker.

Then, 360 mL of solC of gold nanoparticles obtained in example 9.2. are added in this medium under strong stirring. The number of moles of macromolecules corresponds to 12 times the equivalent number of moles of surface gold atoms. The nanoparticles are incubated in the presence of polymer for at least 12 hours.

9.3.4 Purification of Functionalized 18 nm Gold Nanoparticles.

The objective of this operation is to eliminate the excess of hetero-bifunctional PEOs and to concentrate the sol of modified gold nanoparticles in a minimal volume (<5 mL) for a particle concentration higher than 0.1 μM, typically equal to 0.25 μM (1.504.10¹⁷ particles/L), in order to increase the rates of reaction on the surfaces for the next couplings. The volume of dispersion is reduced to 10 mL by water evaporation under reduced pressure at 70° C. using a rotary evaporator. The elimination of the polymer excess can be accomplished by ultracentrifugation (16,000 rpm, 15 min, 4° C.) using an ultracentrifuge (Optima™ of Beckman Coulter™). Several cycles of washing with ultrapure water have to be carried out so that the maximum residual polymer concentration does not exceed 10⁻⁷ mol/L. Purification on exclusion column of Sephadex® type is also possible at this step.

The sol of 18 nm-diameter gold nanoparticles modified by α-amino-ω-mercapto-poly(ethylene oxide) (M_{w}=1737 g/mol) is named solCPN hereafter.

9.4 Coupling of Hetero-Bifunctional PEO (NHS-PEG-Mal) on the Surface of the Modified Gold Nanoparticles of solBPN and solCPN.

The coupling of the hetero-bifunctional PEO NHS-PEG-Mal (M_{w}=3400 g/mol, 85%, Nektar, the USA) is carried out following the same procedure described in example 1.3. After coupling of the polymeric cross-linker, the resulting sols of 4 nm and 18 nm gold nanoparticles are respectively called solBPPmal and solCPPmal hereafter.

A 1 mL volume of solBPN of concentration equal to 3.12 μM of gold nanoparticles prepared according to step 9.3.2 and 1 mL volume of solCPN of concentration equal to 0.25 μM of gold nanoparticles prepared according to step 9.3.4 are diluted in 1 mL of N-(2-Hydroxyethyl)piperazine-N′-(2-ethanesulfonic acid) (HEPES, Sigma) or phosphate buffer of 200 mM concentration at pH 7.2. A mass of 15.5 mg of NHS-PEG-Mal powder is directly added to each solution under vigorous stirring (1200 rpm with the vortex) until complete dissolution of the polymer. The reaction is left reacting for 2 hours at room temperature, under low stirring.

The purification of the nanoparticles is carried out by centrifugation according to the protocol described in 9.3.2 and 9.3.4. After elimination of the supernatant, the pellet is redispersed in 50 mM HEPES or phosphate buffer, which contains 10 mM EDTA, adjusted to pH 7, degassed under vacuum and added with argon. Several cycles of washing are carried out so that the maximum residual concentration of NHS-PEG-Mal in the solAPPmal is lower than 10⁻⁸ M. The volume of the solBPPmal is brought back to 1050 μL for a concentration of 2.23 μM of 4 nm gold nanoparticles and the volume of the solCPPmal, to 1000 μL for a concentration of 0.118 μM of 18 nm gold nanoparticles.

9.5 Coupling of Anx5-SH to the Functionalized Gold Nanoparticles of solBPPmal and solCPPmal.

The coupling of the double mutant Annexin-A5 [T163C; C314S] to gold nanoparticles of solBPPmal and solCPPmal was achieved using the same procedure described in 1.4. Anx5-SH monomer is obtained by reduction of Anx5-S—S-Anx5 dimers by dithiothreitol (DTT, Sigma) and purification by anion exchange chromatography.

A volume of 525 μL of solBPPmal of concentration equal to 2.23 μM of particles prepared according to example 9.4 is added to 46.6 μL of Anx5-SH solution (1.52 mg/mL) under stirring with a vortex. For the 18 nm gold nanoparticles of the solCPPmal, a volume of 500 μL with a concentration of 0.25 μM of particles is added to 46.9 μL of Anx5-SH solution (1.52 mg/mL). Each tube is closed under inert atmosphere (argon) and the reaction is left for at least 12 h at room temperature. The quantity of Anx5 used for the coupling corresponds to 5 times the theoretical quantity of annexin necessary to cover totally the surface developed by the gold nanoparticles. The resulting suspensions of Anx5-functionalized gold nanoparticles are called solBPP-A5 and solCPP-A5 hereafter.

Purification is carried out by centrifugation or by ultrafiltration according to the protocol describes in example 1.2.3. After elimination of the supernatant, the pellets are redispersed in 10 mM HEPES, pH 7.4, 150 mM NaCl, 2 mM NaN₃. After 4 cycles of washing, solBPP-A5 particles of concentration equal to 4.145 μM of particles (namely 2.49 10¹⁸ particles/L) are diluted to 0.829 μM of particles (5 10¹⁷ particles/L) in a volume of 1 mL of the same buffer and are stored in weak adhesion microtubes at 4° C. After the washing step, the solCPP-A5 is diluted from 0.525 μM to 0.1 μM by adding 1 mL of the buffer and stored in weak adhesion microtubes at 4° C. These sols are stable in physiological medium, in the presence of calcium ions and do not present any sign of flocculation (i.e. colloidal destabilization) after several months of storage.

The same protocol is used for coupling Anx5-ZZ molecules to functionalized gold nanoparticles of solAPPmal. Two mutant Anx5 molecules were used: [T163C; C314S] and [A260C; C314S], leading to almost identical results.

### EXAMPLE 10

### Comparison of the Binding of solAPP-A5, solBPP-A5 and solCPP-A5 Gold Nanoparticles to Supported Lipid Bilayers, by QCM-D

The binding of Anx5-coupled gold nanoparticles (solAPP-A5, solBPP-A5 and solCPP-A5) prepared according to examples 1.4 and 9.5 to supported lipid bilayers containing PS was measured in a quantitative manner by the QCM-D method (37).

FIG. 11 represents QCM-D measurements of the binding of solAPP-A5, solBPP-A5 and solCPP-A5 to a (PC/PS, 4:1) supported lipid bilayers. The mass values obtained at saturation, namely 2 μg, 3.64 μg and 7.1 μg for solAPP-A5, solBPP-A5 and solCPP-A5 respectively, are close to the values of 2.07 μg, 3.6 μg and 8.67 μg, predicted by considering a close-packed assembly of particles. These results demonstrate that Anx5-functionalized gold nanoparticles bind at saturation on a PS-containing lipid bilayers surface.

### EXAMPLE 11

### Control of the Number of Anx5 Protein Per Gold Nanoparticles of solAPP-A5 (10 nm)

The number of Anx5 molecules can be controlled by tuning the amount of protein added to the SolAPPmal during the coupling procedure described in example 1.4. For this, the amount of Anx5 protein has been optimized by gel electrophoresis experiments (polyacrylamide gel electrophoresis, PAGE) in denaturing conditions (in presence of sodium dodecyl sulphate, SDS). FIG. 12 shows that a maximum amount of 10 Anx5 molecules can be coupled per gold nanoparticle of solAPPmal.

The QCM-D experiment presented in FIG. 13 shows that when the amount of Anx5 added to the solAPPmal in the step described in example 1.4 is decreased by 10×, the number of Anx5 coupled per gold nanoparticle of solAPPmal is close to 1.

The binding of the Anx5-gold nanoparticles conjugates of solAPP-A5 in 1/10 condition is 1) specific, their binding being reversed by addition of the calcium chelating agent ethylene-bis(oxyethylenenitrilo)tetraacetic acid (EGTA), 2) saturating, and 3) the Anx5-gold nanoparticles bound to a supported lipid bilayer nanoparticles are not able to bind PS-containing LUVs, in agreement with the fact that one single Anx5 molecule is bound per gold nanoparticle. The mass of Anx5-coupled gold nanoparticles bound to the supported lipid bilayer at saturation is close to 3.6 μg; this value is the same as that obtained with solAPP-A5 in 1/1 coupling conditions described in example 1.4. This result shows that binding of Anx5-functionalized gold nanoparticles to PS-containing supported lipid bilayers is independent of the number of Anx5 molecules per gold nanoparticle is sufficient.

### EXAMPLE 12

### Control of the Number of Anti-PY79 Spore Antibodies Bound to solAPP-A5-ZZ Gold Nanoparticles (10 nm)

The amount of antibodies coupled to gold nanoparticles of solAPP-A5-ZZ described in example 1.4 can be controlled by adjusting their concentration during the step of addition to the solAPPmal. The PAGE experiments shown in FIG. 14 allow to determine the saturating condition (1/1) of antibody PY79 anti α-spores coupled to the gold nanoparticles of solAPPmal in non denaturing conditions (FIG. 14 A). This saturating condition corroborate with that determined for the solAPP-A5 in example 11 (i.e. 10 Ab/nanoparticles of solAPP-A5-ZZ); the PAGE performed with the gold nanoparticles of solAPP-A5-ZZ for different amount of anx5-ZZ after removing to the excess of antibody PY79 anti α-spores by centrifugation, in denaturing conditions (FIG. 14 B) shows the corresponding amounts of antibody liberated to the nanoparticle surface and revealed by the coomassie blue. This PAGE allows verifying the saturating condition for the 1/1 coupling condition.

### REFERENCES

1—Beesley J E. in “Colloidal gold: principles, methods and applications” M A Hayat, ed., Academic Press, New York, 1989, Vol. 1, pp 421-425.
2—Hayat M A, “Immunogold-silver staining. Principles, methods and applications”. 1995, pp 1-336.
3—Edmund Gutierrez, Richard D. Powell, James F. Hainfeld and Peter M. Takvorian, A Covalently Linked 10 nm Gold Immunoprobe Proceedings of the fifty-seventh Annual Meeting, Microscopy Society of America; G. W. Bailey et al. (Eds.) Springer-Verlag, New York, N.Y., 1999, pp. 1324-1325
4—Otsuka H, Akiyama Y, Nagasaki Y, Kataoka K. Quantitative and reversible Lectin-Induced association of gold nanoparticles modified with α-lactosyl-ω-mercapto-poly(ethylene glycol). J. Am. Chem. Soc. 2001, 123:8226-8230.
5—Templeton A C, Chen S, Gross S M, Murray R W. Water-Soluble, Isolable Gold Clusters Protected by Tiopronin and Coenzyme A Monolayers. Langmuir 1999, 15:66-76.
6—Templeton A C, Cliffel D E, Murray R W. Redox and Fluorophore Functionalization of Water-Soluble, Tiopronin-Protected Gold Clusters. J. Am. Chem. Soc. 1999, 121:7081-7089.
7—Abad J M, Mertens S F L, Pita M, Fernandez V M, Schiffrin D J. Functionalization of thioctic acid-capped gold nanoparticles for specific immobilization of histidine-tagged proteins. J. Am. Chem. Soc. 2005, 127; 5689-5694.
8—Roux S, Garcia B, Bridot J L., Salome M, Marquette C, Lemelle L, Gillet P, Blum L, Perriat P, Tillement O. Synthesis, characterization of dihydrolipoic acid capped gold nanoparticles, and functionalization by the electroluminescent luminol. Langmuir 2005, 21:2526-2536.
9—Levy R, Thanh N T K, Doty R C, Hussain I, Nichols R J, Schiffrin D J, Brust M, Fernig D G. Rational and combinatorial design of peptide capping ligands for gold nanoparticles. J. Am. Chem. Soc. 2004, 126:10076-10084.
10—Zhenxin Wang, Raphaël Lévy, David G. Fernig and Mathias Brust, The Peptide Route to Multifunctional Gold Nanoparticles, Bioconjugate Chem., 16 (3), 497-500, 2005.
11—de la Fuente J, Berry C C. Tat peptide as an efficient molecule to translocate gold nanoparticles into the cell nucleus. Bioconjugate Chem. 2005, 16:1176-1180.
12—Tkachenko A G, Xie H, Liu Y, Coleman D, Ryan J, Glomm W R, Shipton M K, Franzen S, Feldheim D L. Cellular trajectories of peptide-modified gold particle complexes: comparison of nuclear localization signals and peptide transduction domains. Bioconjugate Chem. 2004, 15, 482-490.
13—Shenoy D, Fu W, Crasto C, Jones G, Dimarzio C, Sridhar S, Amiji M. Surface functionalization of gold nanoparticles using hetero-bifunctional poly(ethylene glycol) spacer for intracellular tracking and delivery. Int. J. Nanomedecine 2006, 1(1): 51-58).
14—Hainfeld J. F., Furuya F. R., Powell R. D. Small organometallic probes. Patent application US200530207.
15—Gerke V, Creutz C E, Moss S E. Annexins: linking Ca2+ signalling to membrane dynamics. Nat. Rev. Mol. Cell. Biol. 2005, 6:449-461.
16—Huber, R., J. M. Römisch, and E. P. Paques. 1990. The crystal and molecular structure of human annexin V, an anticoagulant protein that binds to calcium and membranes. EMBO J. 9:3867-3874.
17—Richter R, Lai Kee Him J, Tessier B, Tessier C, Brisson A R. On the kinetics of adsorption and two-dimensional self-assembly of annexin A5 on supported lipid bilayers. Biophysical Journal 2005, 89:3372-3385.
18—Kerr J F R, Wyllie A H, Currie A R. Apoptosis: a basic biological phenomenon with wide-ranging implications in tissue kinetics. Brit. J. Cancer 1972, 26:239-257.
19—Reutelingsperger, C. P. Annexins: key regulators of haemostasis, thrombosis and apoptosis. Thromb. Haemost. 2001, 86:413-419.
20—Dachary-Prigent, J., Freyssinet, J.-M., Pasquet, J.-M., Carron, J.-C., and Nurden, A., T. Annexin V as a probe of aminophospholipid exposure and platelet membrane vesiculation: a flow cytometry study showing the role for free sulfhydryl groups. Blood 1993, 81:2554-2565.
21—Vermes, I., Haanen, C., Steffens-Nakken, H. & Reutelingsperger, C. J. Immunol. Methods 1995, 184:39-51.
22—Patent application WO2005114192: A DEVICE FOR BINDING A TARGET ENTITY TO A BAIT ENTITY AND DETECTION METHODS USING THE SAME (A. Brisson).
23—Loewenadler et al., Gene 1987, 58:87.
24—Nilsson et al., Protein Engineering 1987, 1:107.
25—Turkevich J, Stevenson P C, Hillier J. Nucleation and growth process in the synthesis of colloidal gold. Discuss. Faraday Soc. 1951, 11:55-75.
26—Frens G. Controlled Nucleation for the regulation of the particle size in monodisperse gold suspensions. Nature: Phys. Sci. 1973, 241:20-22.
27-Daniel M C, Astruc D. Gold nanoparticles: Assembly, supramolecular chemistry, quantum-size-related properties, and applications toward biology, catalysis, and nanotechnology. Chem. Rev. 2004, 104:293-346.
28—Brust M, Walker M, Bethell D, Schiffrin D, Whyman R. Synthesis of thiol-derivatised gold nanoparticles in a two-phase liquid-liquid system. J. Chem. Soc., Chem. Commun. 1994, 801-802.
29—Brust M, Fink J, Bethell D, Schiffrin D J, Kiely C J. Synthesis and reactions of functionalised gold nano articles. J. Chem. Soc., Chem. Commun. 1995, 1655-1656.
30—Busbee B D, Obare S O, Murphy C J. An improved synthesis of high-aspect-ratio gold nanorods. Adv. Mater. 2003, 15:414-416.
31—Giersig M, Mulvaney P. Preparation of ordered colloid monolayers by electrophoretic deposition. Langmuir 1993, 9:3408-3413.
32—Wuelfing W P, Stephen M G, Miles D T, Murray R W. Nanometer gold clusters protected by surface-bound monolayers of thiolated poly(ethylene glycol) polymer electrolyte. J. Am. Chem. Soc. 1998, 120:12696-12697.
33—Zwaal R F, Schroit A J. Pathophysiologic implications of membrane phospholipid asymmetry in blood cells. Blood 1997, 89:1121-1132.
34—Mallat Z. et al. Shed membrane microparticles with procoagulant potential in human artherosclerotic plaques: a role for apoptosis in plaque thrombogenicity. Circulation 1999, 99:348-355.
35—Demo S D, Masuda E, Rossi A B et al. Quantitative measurement of mast cell degranulation using a novel flow cytometric annexin-V binding assay. Cytometry 1999, 36:340-348.
36—Uhlen et Moks. Methods in Enzymology 1990, 185:129-143.
37—Rodahl M, Hook F, Krozer A, Brzezinski P, Kasemo B. Quartz crystal microbalance setup for frequency and Q-factor measurements in gaseous and liquid environments. Rev. Sci. Instrum. 1995, 66:3924-3930.
38—Sauerbrey G. Verwendung von Schwingquartzen zurWagung dunner schichten and zurMikrowagung. Zeitschrift fur Physik. 1959, 155:206-222.
39—Mornet S, Lambert O, Duguet E, Brisson A. The Formation of Supported Lipid Bilayers on Silica Nanoparticles Revealed by Cryoelectron Microscopy, NanoLetters, 2005, 5:281-285.
40—Dubochet J, Adrian M, Chang J J, Homo J-C, Lepault J, McDowall A W, Schulz P. Cryo-electron microscopy of vitrified specimens, Quart. Rev. Biophys., 1988, 21:129-228.
41—Mauro M J, Druker B J. Targeting BCR-ABL as therapy for CML. Oncologist 2001, 6:233-238.
42—Hayat M A. Principles and techniques of electron microscopy. Biological applications. 4th edition. Cambridge University Press. 2000.
43—Aronson A I, Fitz-James P. Structure and morphogenesis of the bacterial spore coat. Bacteriological Reviews, 1976, 40:360-402.
44—Jana N R, Gearheart L, Murphy C J. Wet chemical synthesis of high aspect ratio cylindrical gold nanorods. J. Phys. Chem. B, 2001, 105(19):4065-4067.

1.-33. (canceled)

34. Surface functionalized nanoparticles, wherein said nanoparticles have a size of between 1 nm and 1 μm and have a surface modified by grafting thereon by covalent linkage a plurality of spacers, said spacers being linked in a stereo-specific manner to a stereo-specifically modified protein to provide controlled orientation of the particle-bound protein.

35. Surface functionalized nanoparticles according to claim 34, wherein said stereo-specifically modified protein is selected from the group consisting of mutant protein, fusion protein, protein modified by addition of a poly-histidine extension and protein modified by addition of a biotin group.

36. Surface functionalized nanoparticles according to claim 34, wherein said stereo-specifically modified protein is a mutant protein in which a cysteine presenting an accessible thiol group has been inserted, said thiol group being accessible for linkage to the nanoparticles via said spacers.

37. Surface functionalized nanoparticles according to claim 34, wherein said stereo-specifically modified protein is selected from the group consisting of stereo-specifically modified annexins, coagulation factors, phospholipases, lactadherin, and proteins containing one or several membrane-binding C2-domains.

38. Surface functionalized nanoparticles according to claim 34, wherein said stereo-specifically modified protein is a mutant annexin in which one single cysteine with an accessible thiol group has been inserted and/or is an annexin derived fusion protein which binds to the Fc fragment of antibodies.

39. Surface functionalized nanoparticles according to claim 34 wherein said stereo-specifically modified protein is a modified annexin selected from the group consisting of Annexin-A1, Annexin-A2, Annexin-A3, Annexin-A4, Annexin-A5, Annexin-A6, Annexin-A7, Annexin-A8, Annexin-A9, Annexin-A12, Annexin-A, Annexin-B, Annexin-C, and Annexin-D.

40. Surface functionalized nanoparticles according to claim 34, wherein said stereo-specifically modified protein is a double mutant Annexin-A5 from Rattus norvegicus containing a C314S mutation and a mutation selected from the group consisting of T163C, A164C, I165C and A2C.

41. Surface functionalized nanoparticles according to claim 34, wherein said stereo-specifically modified protein is a double mutant Annexin-A5 from Rattus norvegicus having the C314S and T163C mutations.

42. Surface functionalized nanoparticles according to claim 34, wherein said stereo-specifically modified protein is an annexin derived fusion protein selected from the group consisting of Annexin-Z fusion protein and Annexin-ZZ fusion protein, where Z is a fragment of protein A from Staphylococcus aureus.

43. Surface functionalized nanoparticles according to claim 42, wherein the Annexin-Z fusion protein and the Annexin-ZZ fusion protein contain Annexin-A5 double mutant from Rattus norvegicus having a double mutation selected from the group consisting of [T163C;C314S], [A260C;C314S], [W185C;C314S], [G259C;C314S], [G261C;C314S], [G28C;C314S], [L29C;C314S], [G30C;C314S], [G1000;C314S], [A101C;C314S], [G102C;C314S], [G186C;C314S] and [T187C;C314S].

44. Surface functionalized nanoparticles according to claim 34 wherein said nanoparticles are selected from the group consisting of gold, silver, platinum, palladium, transition metal chalcogenides passivated by zinc sulfide, iron-gold alloy and iron-platinum alloy nanoparticles.

45. Surface functionalized nanoparticles according to claim 34, wherein said spacers are selected from the group consisting of homo-bifunctional polyethylene oxides, hetero-bifunctional polyethylene oxides, homo- or hetero-bifunctional polyethylene oxide containing linkers, homo- or hetero-polypeptides, and functionalized oligonucleotides.

46. Surface functionalized nanoparticles according to claim 34, wherein said spacers are terminated by a-SH reactive group which is linked to the accessible thiol (—SH) group of a cysteine inserted in said stereo-specifically modified protein, when said spacers are linked by covalent linkage to said stereo-specifically modified protein.

47. Surface functionalized nanoparticles according to claim 34, wherein when said spacers are linked by affinity linkage to said stereo-specifically modified protein, said stereo-specifically modified protein is a protein modified by addition of a poly-histidine extension and said spacers are terminated by a Ni-NTA group which is linked to said poly-histidine extension.

48. Surface functionalized nanoparticles according to claim 34, wherein when said spacers are linked by affinity linkage to said stereo-specifically modified protein, said stereo-specifically modified protein is a protein modified by addition of a biotin group and said spacers are terminated by a biotin group which is linked to a streptavidin, itself linked to the biotin group inserted in said stereo-specifically modified protein.

49. Surface functionalized nanoparticles according to claim 34, wherein said nanoparticles are gold nanoparticles having a size of between 1 nm and 50 nm, wherein said stereo-specifically modified protein is a modified Annexin-A5 and wherein said spacers are homo or hetero bifunctionnal polyethylene oxides covalently linked to said stereo-specifically modified protein.

50. Surface functionalized nanoparticles according to anyone of claim 34 wherein said spacer comprises one or more covalently-linked spacers selected from the group consisting of homo- or hetero-bifunctional polyethylene oxides.

51. Surface functionalized nanoparticles according to claim 34 wherein stereo-specifically modified protein is a mutant protein in which a cysteine presenting an accessible thiol group has been inserted, said thiol group being accessible for linkage to the nanoparticles via said spacer and wherein said spacer consists of two covalently linked homo- or hetero bifunctional polyethylene oxide spacers, the first spacer being covalently linked to said nanoparticles and the second spacer being covalently linked to the first spacer at one end and linked to said stereo-specifically modified protein at the other end, wherein the first homo- or hetero-bifunctional polyethylene oxide (PEO or PEG) spacer has the formula (1)
Nu₁-PEG-Nu₂ (1)
wherein:
Nu₂ represents a nucleophilic group able to be covalently linked to the surface of the nanoparticle, said nucleophilic group being selected from the group consisting of —SH group and other gold reactive groups, and
Nu₁ represents a nucleophilic group selected from the group consisting of —SH, —NH₂ and —OH groups,
and wherein the second homo- or hetero-bifunctional polyethylene oxide spacer presents at one end a group able to react with —SH, —NH₂ and —OH group, and at the other end a thiol reactive group able to react with the thiol group of a cysteine of said stereo-specifically modified protein.

52. Surface functionalized nanoparticles according to claim 34, wherein the nanoparticles are gold nanoparticles functionalized by a first polyethylene oxide spacer containing a terminal thiol group (Nu₂=SH) and a second spacer selected from the group consisting of homo bifunctional polyethylene oxide comprising bis-maleimides (Mal-PEG-Mal), bis-orthopyridyldisulfides (OPSS-PEG-OPSS) and bis-vinylsulfones (VS-PEG-VS).

53. Surface functionalized nanoparticles according to claim 34, wherein the nanoparticles are gold nanoparticles, which are functionalized by a first polyethylene oxide spacer having a molar mass higher than 300 g/mol and containing a terminal thiol group (Nu₂=SH) and the second polyethylene oxide spacer is selected from the group consisting of homo-bifunctional bis-maleimide coupling agents comprising α,\acute{ω}-bis-maleimido(di-, tri- or tetra-) ethyleneglycol.

54. Aqueous dispersion containing surface functionalized nanoparticles according to claim 34.

55. Method for obtaining surface functionalized nanoparticles comprising the following steps:
a) optionally, preparation of the nanoparticles,
b) functionalization of the nanoparticles by fixing a plurality of spacers by a covalent linkage,
c) optionally, purification of the functionalized nanoparticles obtained in step b), in order to eliminate the spacers in excess,
d) coupling on said spacers, by covalent or by affinity linkage, a stereo-specifically modified protein having affinity for anionic phospholipids or other membrane-associated components, and
e) optionally, purification of the functionalized nanoparticles obtained in step d).

56. Method for detecting cells or cell fragments exhibiting a physiological or pathological state involving membrane reorganization with the exposure of phosphatidyl-serine molecules, said method including:
a) coupling the surface functionalized nanoparticles according to claim 34 to the cells or cell fragments;
b) detecting the presence of said functionalized nanoparticles coupled to the cells or cell fragments.

57. Method according to claim 54, wherein the coupling in step a) is made in the presence of calcium ions when said stereo-specifically modified protein is annexin.

58. Method for diagnosing a physiological or pathological state in an individual comprising the following steps:
a) contacting a biological sample of said individual with surface functionalized nanoparticles according to claim 34,
b) detecting and recording whether a complex is formed, and
c) correlating the formation of said complex with a physiological or pathological state.

59. Method according to claim 58, wherein the physiological or pathological state is selected from the group consisting of haematological state, disease involving apoptosis and any state involving membrane reorganization with the exposure of phosphatidyl-serine molecules.

60. Method for detecting a target molecule in a biological sample, comprising the steps of:
a) contacting a biological sample with nanoparticles according to claim 36 which are functionalized with a fusion complex between an Annexin-Z derived fusion protein or an Annexin-ZZ derived fusion protein and an antibody, wherein the Z- or ZZ-domain is linked by affinity to the Fc fragment of the antibody, and wherein said antibody is able to bind with said target molecule,
b) detecting and recording complexes that are formed between the nanoparticles functionalized with the fusion complex and the target molecule when said target molecule is present in said sample, and
c) correlating the formation of said complexes with a physiological or pathological state.
